SEQUENCE LISTING (9 sequences)

SEQ ID NO: 1 - protein, 37 aa, Homo sapiens
DKNTTQHPNVTTLAPISNVTSAPVTSLPLVTTPAPET

SEQ ID NO: 2 - protein, 53 aa, Homo sapiens
SVSTATPVPTANSTAKPTVQPSPSTTSKTVTTSGTTNNTVTPTSQPVRKSTFD

SEQ ID NO: 3 - protein, 195 aa, Artificial Sequence
  MYDGF-164 fusion
VSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTELSVSTATPVPTANSTAKPTVQPSPSTTSKTVTTSGTTNNTVTPTSQPVRKSTFD

SEQ ID NO: 4 - protein, 178 aa, Artificial Sequence
  CD164-MYDGF fusion
DKNTTQHPNVTTLAPISNVTSAPVTSLPLVTTPAPETVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTE

SEQ ID NO: 5 - protein, 230 aa, Artificial Sequence
  FGF21-164 fusion
DSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGPSQGRSPSYASSVSTATPVPTANSTAKPTVQPSPSTTSKTVTTSGTTNNTVTPTSQPVRKSTFD

SEQ ID NO: 6 - protein, 285 aa, Artificial Sequence
  FGFBP3-164 fusion
RREKGAASNVAEPVPGPTGGSSGRFLSPEQHACSWQLLLPAPEAAAGSELALRCQSPDGARHQCAYRGHPERCAAYAARRAHFWKQVLGGLRKKRRPCHDPAPLQARLCAGKKGHGAELRLVPRASPPARPTVAGFAGESKPRARNRGRTRERASGPAAGTPPPQSAPPKENPSERKTNEGKRKAALVPNEERPMGTGPDPDGLDGNAELTETYCAEKWHSLCNFFVNFWNGSVSTATPVPTANSTAKPTVQPSPSTTSKTVTTSGTTNNTVTPTSQPVRKSTFD

SEQ ID NO: 7 - protein, 130 aa, Artificial Sequence
  164-FSHa fusion
DKNTTQHPNVTTLAPISNVTSAPVTSLPLVTTPAPETAPDVQDCPECTLQENPFFSQPGAPILQCMGCCFSRAYPTPLRSKKTMVTSESTCCVAKSYNVAKSYNRVTVMGGFKVENHTACHCSTCYYHKS

SEQ ID NO: 8 - protein, 182 aa, Artificial Sequence
  FSHb-164 fusion
MKTLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKESVSTATPVPTANSTAKPTVQPSPSTTSKTVTTSGTTNNTVTPTSQPVRKSTFD

SEQ ID NO: 9 - protein, 197 aa, Homo sapiens
MSRLSRSLLWAATCLGVLCVLSADKNTTQHPNVTTLAPISNVTSAPVTSLPLVTTPAPETCEGRNSCVSCFNVSVVNTTCFWIECKDESYCSHNSTVSDCQVGNTTDFCSVSTATPVPTANSTAKPTVQPSPSTTSKTVTTSGTTNNTVTPTSQPVRKSTFDAASFIGGIVLVLGVQAVIFFLYKFCKSKERNYHTL

(19) United States
(12) Patent Application Publication
(10) Pub. No.: US 2024/0002469 A1
(43) Pub. Date: Jan. 4, 2024

(54) CD164 FUSION AND USES THEREOF

(71) Applicant: JIANGSU CELL TECH MEDICAL RESEARCH INSTITUTE CO., LTD., Nanjing (CN)

(72) Inventors: Hongping Yin, Nanjing (CN); Meijia Yang, Scituate, MA (US); Yinghua Miao, Nanjing (CN); Qingchao Ji, Nanjing (CN)

(73) Assignee: JIANGSU CELL TECH MEDICAL RESEARCH INSTITUTE CO., LTD., Nanjing (CN)

(21) Appl. No.: 18/037,257

(22) PCT Filed: Nov. 8, 2021
(86) PCT No.: PCT/CN2021/129378

§ 371 Date: May 16, 2023

(30) Foreign Application Priority Data
Nov. 16, 2020 (WO) PCT/CN2020/129067

(51) Int. Cl. C07K 14/705; C07K 14/59; C07K 14/50; C07K 14/475; A61P 9/10; A61P 13/12

(52) U.S. Cl. CPC C07K 14/70596; C07K 14/59; C07K 14/50; C07K 14/475; A61P 9/10; A61P 13/12; C07K 2319/31; A61K 38/00

(57) ABSTRACT

Provided are CD164 mucin domain fusion proteins with a heterologous protein, such as a heterologous protein with a therapeutic function and can benefit from extended serum half-life. Methods of using the fusion proteins are also provided.

### BACKGROUND OF THE INVENTION

Cell surface mucins are large transmembrane glycoproteins involved in diverse functions ranging from shielding the airway epithelium against pathogenic infection to regulating cellular signaling and transcription. Unlike large mucin proteins, CD164 contains small mucin-like domains less than 60 amino acids in length. So far, no function of mucin proteins had been clearly demonstrated for CD164. Mice deleted of CD164 gene are viable and demonstrated no obvious phenotype. The human CD164 is a member of sialylated, mucin-like membrane proteins with adhesive properties. It is a type I membrane protein with a nearly ubiquitous tissue distribution. It is predominantly located intracellularly within endosomes and lysosomes (Ihrke 2000, Chan 2000). Functional analysis suggested that CD164 may play an accessory role for docking CD34+ cells to the stromal tissue of bone marrow. It contains two mucin domains (I and II) linked by a Cys-rich non-mucin domain, followed by a transmembrane and an intracellular domain (Doyonnas 2000). The mucin domain I of CD164 is comprised of 37 amino acids, putatively modified post-translationally on three N-linked glycosylation and nine O-linked glycosylation sites.

Several monoclonal antibodies against the mucin domain I had been found to bind glyco-epitopes and blocked CD34⁺ cells to attach to bone marrow stromal reticulocytes. Removal of sialic acids reduced the attachment, thus the end-glycosylation such as sialylation might be important for maintaining “stem cell-like” characteristics for CD34⁺ cells (Doyonnas 2000). In addition, CD164 may be involved in migration responses related to CXCR4 signaling (Fordes 2007). In yet another assay system, CD164 Fc fusion produced from transfected 293T cells inhibited multinucleate myotube formation in response to differentiation signals (Lee et al., 2001).

In all cases the CD164 functions were sensitive to the treatment by sialidase or O-glycopeptidase treatment, suggesting that end glycoforms are required for intercellular signaling and binding.

The mucin domain II is composed of 56 amino acids, putatively modified post-translationally on two N-linked glycosylation and twenty-three O-linked glycosylation sites. There are no evidences to suggest that the epitopes in the mucin domain II are functionally involved in binding. In contrast, CD164 transcript analysis identified additional splicing variants with exon 4 or exon 5 deletions, which removed large parts of the mucin domain II without any effect (Chan 2001). Therefore the domain H forms a brush stem that support a canopy-like structure comprised of the mucin domain I and the cysteine rich region involved in cell-cell interactions.

The cysteine-rich domain separating the mucin domains I and II is comprised of 52 amino acids with 8 cysteine residues capable of forming disulfide bridges. This domain also contains four putative N-linked glycosylation sites, and possibly additional O-linked glycosylation sites.

Together, glycosylation of CD164 may contribute to 70% of the molecular mass if fully glycosylated. Observation of 90 kDa glycosylated forms of CD164 from human bone marrow cells, CD34⁺ purified cord blood cells, or cultured bone marrow stromal reticular cells support the notion that significant glycosylation were added to the polypeptide of 174 amino acid residues of the mature form of the human CD164 (Doyonnas 2000).

### SUMMARY OF THE INVENTION

One aspect of the invention provides a fusion protein, comprising: (1) a polypeptide comprising an amino acid sequence having at least 90% identity to SEQ ID NO: 1 or 2, and (2) a heterologous polypeptide.

In certain embodiments, the polypeptide consists of the amino acid sequence of SEQ ID NO: 1 or 2.

In certain embodiments, (1) is C-terminal to (2), optionally, (1) is SEQ ID NO: 2.

In certain embodiments, (1) is N-terminal to (2), optionally, (1) is SEQ ID NO: 1.

In certain embodiments, the fusion protein further comprises (3) a second polypeptide comprising an amino acid sequence having at least 90% identity to SEQ ID NO: 1 or 2, wherein (1) and (3) each comprising a different one of SEQ ID NO: 1 and 2.

In certain embodiments, the fusion protein comprises the polypeptide of SEQ ID NO: 1 fused N-terminal to the heterologous polypeptide, and the polypeptide of SEQ ID NO: 2 fused C-terminal to the heterologous polypeptide.

In certain embodiments, said heterologous polypeptide is a therapeutic polypeptide.

In certain embodiments, the therapeutic polypeptide has a (human or mouse) serum or circulation half-life that is at least 10%, 20%, 30%, 40%, 50%, 60%, 70%, 80%, 85%, 90%, or 95% less than that of the fusion protein.

In certain embodiments, the heterologous polypeptide is fibroblast growth factor 21 (FGF21), follicle-stimulating hormone (FSH), myeloid-derived growth factor (MYDGF), fibroblast growth factor binding protein 3 (FGFBP3), natriuretic peptides B, cholecystokinin, glucagon-like peptide-1 (GLP-1), gonadotropin-releasing hormone, secretin, leuprorelin, enfuvirtide, glucagon, bivalirudin, sermorelin, corticotropin tetracosapeptide, insulin-like growth factor (IGF), parathyroid hormone, or amylin.

In certain embodiments, the fusion protein comprises an O- and/or an N-linked glycosylation.

In certain embodiments, the fusion protein comprises sialylation.

In certain embodiments, the fusion protein further comprises a linker peptide between (1) and (2).

In certain embodiments, in the fusion protein, (1) is C-terminal to (2) and is optionally SEQ ID NO: 2, and the heterologous polypeptide is MYDGF or a functional fragment thereof.

In certain embodiments, the fusion protein has an amino acid sequence having at least 90% identity to any one of SEQ ID NOs: 3-8.

In certain embodiments, the fusion protein has the amino acid sequence of SEQ ID NO:3.

Another aspect of the invention provides a polynucleotide encoding the fusion protein of the invention.

In certain embodiments, the polynucleotide is codon-optimized for expression in a target host cell.

In certain embodiments, the target host cell is a human cell, a rodent cell (e.g., a mouse cell), or a non-human mammalian cell.

Another aspect of the invention provides a vector comprising the polynucleotide of the invention.

In certain embodiments, the vector is an expression vector.

In certain embodiments, the vector is a plasmid.

Another aspect of the invention provides a host cell comprising the fusion protein of the invention, the polynucleotide of the invention, or the vector of the invention.

In certain embodiments, the host cell is a tissue culture cell.

In certain embodiments, the host cell is a CHO K-1 cell (ATCC #CCL61) or a CHO DG44 cell or a CHO DXB-11 cell, a Namalwa cell (e.g., ATCC #CRL-1432), a HeLa cell (ATCC #CCL-2), a HEK293 cell (ATCC #CCL-1573), a WI-38 cell (ATCC #CCL-75), a MRC-5 cell (ATCC #CCL-171), a HepG2 cell (ATCC #HB-8065), a 3T3 cell (ATCC #CCL-92), a L-929 cell (ATCC #CCL-1), a Myeloma (e.g., NS/O) cell, a BHK-21 cell (ATCC #CCL-10), a COS-7 cell (ATCC #CCL-1651), or a Vero cell (ATCC #CCL-81), or a derivative thereof.

In certain embodiments, the host cell is a CHO K-1 cell (ATCC #CCL61) or a derivative thereof, or a HEK293 cell (ATCC #CCL-1573) or a derivative thereof.

Another aspect of the invention provides a pharmaceutical composition comprising a therapeutically effective amount of the fusion protein of the invention, the polynucleotide of the invention, or the vector of the invention, and a pharmaceutically acceptable additive or excipient.

In certain embodiments, the pharmaceutical composition is formulated for intravenous injection.

Another aspect of the invention provides a method of enhancing serum/circulation half-life for a protein, comprising fusing the protein to a polypeptide comprising an amino acid sequence having at least 90% identity to SEQ ID NO: 1 or 2.

In certain embodiments, the polypeptide consists of the amino acid sequence of SEQ ID NO: 1 or 2.

In certain embodiments, the protein is fused N-terminal to SEQ ID NO: 2.

In certain embodiments, the protein is fused to the polypeptide via a linker polypeptide.

Another aspect of the invention provides a method of treating a disease, disorder, or condition in a subject in need thereof, the method comprises administering to the subject a therapeutically effective amount of the fusion protein of the invention, the polynucleotide of the invention, or the vector of the invention, wherein the disease, disorder, or condition is treatable by said heterologous polypeptide.

In certain embodiments, the disease, disorder, or condition is selected from the group consisting of a tissue injury, a cardiovascular disease, an inflammatory disease or disorder, and a kidney disease.

In certain embodiments, the tissue injury is an acute injury such as myocardio infarction or stroke.

In certain embodiments, the tissue injury is a chronic injury such as diabetic injury to kidney.

In certain embodiments, the cardiovascular disease is selected from the group consisting of myocardial infarction, arteriosclerosis, hypertension, angina pectoris, hyperlipidemia, and heart failure.

In certain embodiments, the inflammatory disease or disorder is selected form the group consisting of Type I diabetes, Type II diabetes, pancreatitis, nonalcoholic fatty liver disease (NAFLD), and nonalcoholic steatohepatitis (NASH).

In certain embodiments, the disease or disorder is a kidney disease.

In certain embodiments, the subject is a human.

### BRIEF DESCRIPTION OF THE DRAWINGS

FIG. 1 shows Western blot detection of glycosylated forms of CD164 mucin domain fusion proteins. MYDGF-164 and FGF21-164 were expressed in 293F cells. Conditioned medium were collected and analyzed by SDS-PAGE and Western blot. “R” designates samples that were reduced by DTT.

FIG. 2 is Western blot detection of naturally glycosylated proteins after fusion to CD164 mucin domains. 164-FSHa or FSHa, and FSHb-164 or FSHb) were stably transfected into CHO cells, along with their respective heterodimeric subunits. Both non-reduced (NR) and reduced (R) samples were analyzed by SDS-PAGE and Western blot. Same analysis was conducted for FGFBP3-164 expressed in 293F cells.

FIGS. 3A and 3B show purified MYDGF-164 fusion protein analyzed by SDS-PAGE and CBB staining (FIG. 3A) or RP-HPLC (FIG. 3B).

FIG. 4 shows acidic forms of MYDGF-164 fusion upon expression in CHO cells. Purified MYDGF-164 (sample A001) was analyzed by isoelectric focusing (IEF), along with pI markers in its adjacent lane. Predominant isoforms were below pI 5.1.

FIG. 5 shows monosaccharide composition analysis, indicating that GlcNAc:GalNAc:Gal:Man:Fuc in the MYDGF-164 fusion protein is 7:5:11:2:1. A large amount of galactose in the MYDGF-164 fusion protein indicated that it contains O-glycans and N-glycans.

FIG. 6 is β-elimination analysis showing that the content of O-glycans in MYDGF fusion protein was much higher than that of N-glycans.

FIG. 7 is UPLC characterization of N-linked glycan forms derived from MYDGF-164 after PNGase treatment and 2-AB labeling. Elution times of various peaks are indicated in minutes. Glycan forms corresponding to peaks are indicated with pictographs for glycans.

FIG. 8 shows analysis of the content and types of sialic acid in MYDGF-164 fusion protein using UPLC. The analysis showed that MYDGF-F164 fusion protein contains 6.6% Neu5Ac).

FIG. 9 is a MALDI-TOF mass spectrum of MYDGF-164 fusion. M⁺: singly protonated species. M²⁺: doubly protonated species.

FIGS. 10A and 10B show SEC-HPLC analysis of purified MYDGF-164 fusion (FIG. 10A) and standard proteins (FIG. 10B). The molecular weights (MWs) of marker proteins (Thyroglobulin: 67,000; γ-globulin: 150,000: Ovalbumin: 45,000; Myoglobin: 17,000; Angiotensin: 1,000) were plotted versus their elution times, and fitted by a non-linear regression model (R²=0.99898). The hydrodynamic radius of the MYDGF-164 fusion protein was calculated as 98.744 kDa.

FIG. 11 shows pharmacokinetic (PK) analysis of the purified MYDGF-164 fusion injected into C57BL/6 mice per i.v. administration. The concentration of MYDGF-164 fusion in sera were analyzed by LC-MS/MS.

FIG. 12 shows that HUVEC cell proliferation was enhanced by the MYDGF-164 fusion protein when co-incubated with 5% fetal bovine serum (FBS). Addition of MYDGF-164 to HUVEC cells in 5% FBS demonstrated a dose-dependent effect on cell proliferation in 5% FBS. ***P<0.001, **P<0.01, *P<0.05 using one-way ANOVA.

FIG. 13 shows that MYDGF-164 facilitated cell cycle activities of HUVEC cells in 1% fetal bovine serum (Left). Proportions of cells in different cell cycle phases were shown as bar graph (right). ***P<0.001, **P<0.01, *P<0.05 using one-way ANOVA.

FIG. 14 shows scratch recovery analysis to demonstrate that MYDGF-164 enhanced the cellular migration. Monolayer of HUVECs were scratched mechanically, tracks of scratch were repaired by cellular migration. Scratch repair for a monolayer of endothelial cells was tested in the presence of different concentrations of MYDGF-164 or 100 ng/ml VEGFA. Images of monolayer were captured at 0, 12 and 24 hrs. The migration rate was analyzed using the Image-Pro-Plus program. The migration rate is defined as changing area/wound area. ***P<0.001, **P<0.01, *P<0.05 using one-way ANOVA.

FIG. 15 shows that tube formation by HUVEC cells in growth factor-reduced matrigel was enhanced by MYDGF-164 or 100 ng/ml VEGFA after 4-hr incubation. Endothelial tubes were defined closed tubes as circular structures surrounded by tube cells, and the ex the number of closed tubes. ***P<0.001, **P<0.01, *P<0.05 using one-way ANOVA.

FIG. 16 shows that hydrogen peroxide induced HUVEC cell apoptosis was reduced by MYDGF-164. HUVEC cells were preincubated with 1 μg/ml MYDGF-164 for 24 hours before treatment with H₂O₂ (400 μmol/L). Apoptosis or cell death was assayed with annexin V-FITC or propidium iodide staining followed by flow cytometry. ***P<0.001, **P<0.01, *P<0.05 using one-way ANOVA.

FIG. 17A and FIG. 17B depict schematics of experimental plan testing protective function of MYDGF-164 in a rat myocardial ischemia model. In the first ischemic experimental plan (FIG. 17A), MYDGF-164 was administered 5 min before and 4 hours after reperfusion, followed by twice a day intravenous injections for 7 consecutive days before animals were assessed for infarct area quantification. Tirofiban, a non-peptidal antagonist of the GP IIb/IIIa receptor that prevents platelet aggregation was used as the positive control. In the second ischemic experimental plan (FIG. 17B), MYDGF-164 was administered 5 min before and 4 hours after reperfusion, followed by two intravenous injection at 6 and 12 hours post reperfusion and analysis of the infarct sizes at 24 hours. In both experimental plans, cardiac troponin I (cTnI), a biomarker for injuries to the heart muscles after ischemic event was analyzed.

FIG. 18A and FIG. 18B show reproducibly the reduction of myocardial ischemia indicator cardiac-specific troponin I by MYDGF-164 in two independent experiments conducted by two different research organizations. Positive control used was the anti-platelet drug Tirofiban. Release of cTnI in MYDGF-164 treated rats reduced to the levels of the sham-operated rats. Two-way ANOVA: ***p<0.001 model vs. Sham, ###p<0.001 treatment vs. Model.

FIGS. 19A-F show the therapeutic effect of the MYDGF-164 in the rat ischemic model. 19A and 19B show that the infarct area was reduced upon MYDGF-164 treatment based on triphenyl tetrazolium chloride (TTC) staining and infarct area of rat heart after 7 days was determined by excluded darker staining of sectioned heart organ (FIG. 19A). In an independent rat ischemic model, area at risk and infarct area of rat heart after 24 hours were determined by excluded darker staining by both triphenyl tetrazolium chloride (TTC) and Evans blue (FIG. 19D-E). Area at risk were calculated as percentage area of the left ventricle (LV). Infarct area were calculated as percentage area at risk. One-way ANOVA, Tukey's multiple comparisons test. ****P<0.0001 vs. Sham, ***P<0.001 vs Sham, ###P<0.001 vs. MI/R Model, ##P<0.01 vs MI/R Model. FIG. 19F show presence of newly-formed capillaries in the border area between the infarcted and non-infarcted regions. FIG. 19G-H show presence of higher intensity of capillaries in the border region in MYDGF-164 treated rats in two independent experiments.

FIG. 20 shows MYDGF-164 treatment added to survival advantages after myocardial infarction.

FIG. 21 depicts a schematic of experimental plan testing function of MYDGF-164 protecting subjects from renal failure.

FIG. 22 shows serum urea and creatine in adenine-induced injury model rats were reduced by MYDGF-164 treatment. ***P<0.001, **P<0.01, *P<0.05 using one-way ANOVA.

FIGS. 23A-23G show that the MYDGF-164 protein protected renal structural damage induced by adenine. FIG. 23A shows histological images stained with H&E of kidney tissue, showing deposition of symmetric crystalline structures in a tubular lumen (arrow 1), glomerular atrophy (arrow 2), necrosis tubules (arrow 3), cast (arrow 4) and inflammation (arrow 5). Scale bars, 250 μm. In FIG. 23B, the ratio of kidney weight to body weight, or relative kidney weight were determined after treatment. N=5. Lesions scores by 5-point method for various kidney tissue damages were shown in graphs: Tubular necrosis (FIG. 23C), Glomerular atrophy (FIG. 23D), Inflammation (FIG. 23E), Tubular dilation (FIG. 23F), and Pigmentation (FIG. 23G). The 5-point method for determining the degree of lesions follows the following criteria: 1) slight, involving range <10%; 2) mild, involving range 11-25%; 3) moderate, involving range 26-50%; 4) severe, involving range 51-75%; 5) Severe, involving a range of 76-100%. ***P<0.001, **P<0.01, *P<0.05 using one-way ANOVA.

FIGS. 24A-24D show histochemistry analysis of kidney tissues, with representative images on the left panels, and proportions of characterized cells calculated using Image-Pro Plus 6.0 and shown as bar graphs on the right panels. FIG. 24A shows IHC of tubular KIM-1 staining in the paraffin sections from kidney tissues. Scale bars, 100 μm. FIG. 24B shows immunohistochemistry (IHC) analysis using RECA-1 staining in the paraffin sections from kidney tissues. Black arrowheads indicate maintained lumen structures of peritubular capillaries (PTCs), and red arrows indicate collapsed lumen structures of PTCs. Scale bars, 100 μm. FIG. 24C shows IHC of Ki-67 staining in the paraffin sections from kidney tissues. Scale bars, 250 μm. FIG. 24D shows TUNEL assays of apoptotic cells in the paraffin sections from kidney tissues. Scale bars, 50 μm.

FIGS. 25A-25C show that addition of MYDGF164 to HUVEC cells stimulated cellular proliferation (FIG. 25A) and activated MAPK1/3 phosphorylation (FIG. 25B) in a dose-dependent manner. In addition, cyclin D1 expression was enhanced by MYDGF164 fusion (FIG. 25C)

FIGS. 26A-26D show that MYDGF164 is superior to Nicorandil in driving HUVEC cell migration in a scratch assay. In this assay, HUVEC cell monolayer was scratched before treatment of MYDGF164 or Nicorandil for 24 hours. Representative photomicrographs taken at 100× magnification (scratch repair distances indicated with the black double arrow) (FIG. 26A and FIG. 26C). Migration rate of HUVECs were determined after treatment with MYDGF164 or Nicorandil (FIG. 26B and FIG. 26D), N=3, ***P<0.001, *P<0.05 using two-way ANOVA with Tukey's multiple comparisons test.

FIGS. 27A-27B show that 4-hour tube formation of HUVECs after stimulation with MYDGF164 was more enhanced compared to treatment with Nicorandil. VEGFA was used as a positive control. Representative photomicrographs taken at 40× magnification (FIG. 27A). Quantification of closed tubes in per field of HUVECs after treatment with MYDGF164, Nicorandil, or VEGFA (FIG. 27B), N=3, ***P<0.001, *P<0.05 using one-way ANOVA with Tukey's multiple comparisons test.

FIG. 28 show the purified FGF21-164 fusion protein that were stably expressed in 293F cells. Left: SDS-PAGE and CBB staining of the purified FGF21-164 fusion. Right: Western blot analysis.

Method: Protein purities were determined by SDS-PAGE with the Mini-PROTEAN® Tetra System (Bio-Rad) followed by densitometry after Coomassie Brilliant Blue (CBB) staining. The molecular weight standard (11-180 kDa, Tanon) was applied for analysis. For purity analysis, about 10 ug fusion proteins were solubilized in sample buffer and resolved in 12% SDS-PAGE gel, and densitometry was performed using Tanon 4600SF gel image analysis system.

FIG. 29 show the data from the MALDI-TOF analysis of purified FGF21-164 fusion protein. In the analysis, MALDI-TOF mass peak of 42751.5 Da were singly protonated FGF-164 protein, and the peak of 21601.173 Da was doubly protonated protein.

Method: Mass spectra were acquired using the Bruker Autoflex Speed instrument (equipped with a 1000 Hz Smartbeam-II laser) and 2,5-Dihydroxybenzoic acid was used as matrix. The mass spectra of FGF21-164 were analyzed using Bruker Flexanalysis software version 3.3.80.

FIG. 30A-C show data from analytical SEC analysis of the purified FGF21-164 protein. A. FGF21-164 migrated as a single symmetrical peak in SEC analysis. B. Retention time of various reference proteins during the SEC analysis. The molecular weights (MWs) of marker proteins (Thyroglobulin: 67,000; 7-globulin: 150,000; Ovalbumin: 45,000; Myoglobin: 17,000; Angiotensin: 1,000) were plotted versus their elution times. C. Regression analysis of the retention times versus the molecular weight of the reference proteins, and fitting used a non-linear regression model. The hydrodynamic radius of the FGF-164 fusion protein was calculated as 87.679 kDa.

FIG. 31A. UPLC characterization of N-linked glycan forms derived from FGF21-164 after PNGase treatment and 2-AB labeling. Elution times of various peaks are indicated in minutes. Glycan forms corresponding to peaks are indicated with pictographs for glycans. FIG. 31B is β-elimination analysis showing the content of O-glycans in FGF21-164 fusion protein.

Method: O-glycans and N-glycans were released from samples using an improved β-elimination. Proteins and salts were removed using a graphitized carbon cartridge (Supelclean™ ENVI™-Carb SPE) that was equilibrated with water, glycans were eluted with 20% and 40% acetonitrile. The eluents were evaporated by vacuum. Glycans were fluorescence-labeled with 2-aminobenzamide (2-AB) and the labeled glycans were separated on HILIC UPLC (ACQUITY UPLC Glycan BEH Amide Column, 130 Å, 1.7 μm, 2.1 mm×150 mm).

N-linked glycans were labeled with 2-aminobenzamide after PNGase F treatment according to the manufacturer's protocol (Sigma). Labeled glycans were separated on HILIC UPLC (ACQUITY UPLC Glycan BEH Amide Column, 130 Å, 1.7 μm, 2.1 mm×150 mm) to determine the forms of the N-linked glycans based on comparison to the 2-AB labeled dextran glycan standards.

FIG. 32. Pharmacokinetic analysis of the FGF21-164 fusion after intravenous injection into C57/BL6 mice. Serum concentration of the FGF21-164 protein over the time was determined by LC-MS.

Method: LC-MS analysis was used to determine the in vivo half-life of the FGF21-164 fusion protein using the FGF21-164 peptide YLYTDDAQQTE AHLEI (YLY peptide). C57/BL6 mice were injected FGF21-164 protein (22.7 mg/kg) intravenously, serum samples were prepared at various timepoint after injection, and treated with trypsin to release the YLY peptide. LC-MS was used to quantify the concentration of the YLY peptide to trace the FGF21-164 protein.

FIG. 33. Stimulation of glucose uptake by FGF21-164 fusion protein. Glucose concentration in the condition medium was determined after differentiated 3T3-L1 adipocytes were incubated with various concentration of FGF21-164.

Method: Cultured 3T3-L1 adipocytes was pre-treated with high-glucose DMEM with 0.1% FBS for 24 hours, media was changed regular medium with additions of various concentration of FGF21-164. Concentration of glucose in the medium was determined after incubation for 24 hours. Glucose uptake stimulated by FGF21-164 was significant (*p<0.05, ***p<0.001, ****p<0.0001 using one-way ANOVA with Dunnett's multiple comparisons test).

FIG. 34. Reduction of serum glucose in ob/ob mice FIG. 34A. The ob/ob mice demonstrated high baseline levels of glucose levels even after overnight starvation. FGF21-164 fusion reduced serum glucose levels once administered into mice. FIG. 34B. Glucose levels for treatment group showed trend of higher reductions.

Method: Male mice of the ob/ob mouse strain B6/JGpt-Lepem1Cd25/Gpt were purchased and treated according to IACUC guidelines of the China Pharmaceutical University. Unless tests were conducted on the mice food were provided ad libitum. Mice were randomly assigned into control (n=4), 6 mg/kg FGF21-164 (low dose, n=5) treatment, and 12 mg/kg FGF21-164 (high dose, n=5) treatment groups. Following overnight food withdrawal, mice were injected subcutaneously PBS for the control group, or FGF21-164 fusion proteins. Blood glucose levels at various time point were determined by sampling through tail vein phlebotomy followed by quantitation using a glucose meter. Differences between the control and FGF21-164 was tested using one-way ANOVA with Tukey's multiple comparisons.

FIG. 35A. 6 micrograms of purified FSH164 fusion protein was analyzed on SDS-PAGE and CBB staining. FIG. 35B. Western blot analysis of the purified FSH164 fusion protein using antibody against glycol-hormone alpha subunit.

FIG. 36. In vitro stimulation of progesterone synthesis in KGN cells after FSH164 incubation.

Method: KGN cells were grown in DMEM/F12+10% FBS+1% P/S, and were seeded into 96-well plate with 2×10⁴ cells per well. Cells were grown for 24 hours and switched to low serum (1% FBS) medium for another 24 hours. Different concentrations of recombinant human FSH or FSH-CD164 were added into the starvation medium with a final volume equal to 150 microliter. After 72 hours, culture supernatant was collected and progesterone biosynthesis was measured by ELISA (DRG).

FIG. 37. FSH164 fusion show extended half-life in vivo compared to FSH

Method: Female, 6-week old immature SD rats (180-190 grams) were used for pharmacokinetic analysis. rh-FSH (10 micrograms/kg) or FSH164 (18.5 micrograms/kg) were injected into the peritoneal of the rats by s.c. administration. Blood were sampled via retro orbital draw, and sera were prepared and analyzed by using DRG FSH ELISA. Pharmacokinetic parameters were calculated using PK Solver 2.0. A non-compartmental model and linear trapezoidal fitting were applied for analysis of the data.

### DETAILED DESCRIPTION OF THE INVENTION

### 1. Overview

The present invention extended known half-life extension technologies such as Fc fusion, albumin fusions, or pegylation. The methods and compositions described herein are unique, partly because they alter the pKa of the molecules to convert proteins with basic charges to acidic molecules, thus enhancing solubilities, tissue distribution and adsorptions, and increasing the bioavailability of fusion proteins. Furthermore, masking of the fusion proteins by glycosylation can reduce immunogenicity, which is advantageous for biotherapeutics that often require frequent and/or long-term dosing.

As expected, the subject fusing proteins or peptides to small mucin domain with high-levels of glycosylation and sialylation also enhanced the pharmarcokinetic characteristics and bioactivities of biotherapeutics. Therefore, the invention described herein provides a novel technology platform for furnishing biotherapeutics with desired characteristics for use in clinical settings.

More specifically, the invention described herein provides fusion proteins comprising mucin domain I and/or II of (human) CD164 and a heterologous proteins of interest. It was shown that glycosylation and sialylation of the mucin domains were retained when the subject fusion proteins were recombinantly produced, and surprisingly, the half-lives of the fusion proteins were significantly extended compared to that reported in the literature. Thus the invention described herein provides the subject fusion proteins as part of a protein engineering platform useful for optimizing pharmacokinetic (PK) properties of protein therapeutics.

The invention described herein is partly based on the realization that the brush-like structures of mucin domains (due to the O-linked and N-linked glycosylation) can significantly change the hydrodynamic behavior of fusion proteins having such glycosylated domains, and the observation that novel fusion proteins with the highly glycosylated CD164 mucin domains possess improved pharmacokinetic properties, such as greatly enhanced serum half-life and expanded tissue distribution.

While not wishing to be bound by any particular theory, there exists several potential advantages for using the mucin domain fusion proteins of the invention. First, the fusion proteins are highly glycosylated and sialylated, thus reducing the immunogenicity of the fusion proteins. Secondly, sialylation of the fusion proteins may have contributed to the favorable pharmacokinetic properties of the fusion proteins including distribution and adsorptions. Results on IEF, as presented herein, suggest that the subject fusion proteins containing the mucin-like domains are highly acidic, a feature that could improve the solubility of the fusion protein. For example, FGF21 is known to be unstable and forms aggregate at high concentration (Hecht 2012), which may lead to undesired immune responses. Relating to this, FGF21 also has poor tissue distribution, and fusion with the mucin domains of CD164 improves tissue distribution.

Compared to Fc fusion strategy to increase half-life, the CD164 mucin domain fusions of the invention do not have additional effector immune functions that are entailed in the Fc region (such as antibody-dependent cell-mediated cytotoxicity (ADCC) and complement-dependent cytotoxicity (CDC)), thus may be a safer alternative for fusion proteins that bind to cell surface receptors. Also, the Fc fusions are effective approaches that extend the half-life up to days, while the half-life extension by CD164 mucin domains is more tailored to shorter ranges. This is especially useful for therapeutics for which the prolonged overstimulation might be detrimental.

Pegylation strategy had also been previously utilized to extend the half-life of conjugated proteins. According to the invention described herein, however, the cumbersome chemical processes of pegylation is replaced by the natural amino acid polymerization through fusions to CD164 mucin-like domains. Thus the manufacture steps are much more simplified, and the CD164 mucin domain fusions maybe more cost effective in this regard. In addition, mucin domains are more natural compared to the less natural polyethylene moiety, which may cause toxicity after prolonged usage.

Thus in one aspect, the invention provides a fusion protein, comprising: (1) a polypeptide comprising an amino acid sequence having at least 90% identity to SEQ ID NO: 1 or 2, and (2) a heterologous polypeptide.

As used herein, “heterologous polypeptide” refers to a protein or polypeptide that does not originate from a polypeptide or protein comprising SEQ ID NOs: 1 and/or 2. It can be a protein or polypeptide from the same species (e.g., other human proteins/polypeptides) or from a different species.

Within the fusion protein, there is said heterologous polypeptide, the polypeptide comprising an amino acid sequence having at least 90% identity to SEQ ID NO: 1 or 2, and optionally additional sequences such as a linker polypeptide that links the heterologous polypeptide and the polypeptide comprising the amino acid sequence having at least 90% identity to SEQ ID NO: 1 or 2.

In certain embodiments, however, there is no linker between the heterologous polypeptide and the polypeptide comprising the amino acid sequence having at least 90% identity to SEQ ID NO: 1 or 2.

In certain embodiments, the polypeptide comprising the amino acid sequence having at least 90% identity to SEQ ID NO: 1 or 2 consists of the amino acid sequence of SEQ ID NO: 1 or 2. That is, in this embodiment, the fusion protein consists of the heterologous polypeptide, the polypeptide consisting of the amino acid sequence of SEQ ID NO: 1 or 2, and an optional sequence such as a linker that may or may not exist.

The order of the heterologous polypeptide (2) and the polypeptide comprising the amino acid sequence having at least 90% identity to SEQ ID NO: 1 or 2 (1) may be either of the two: (1) is N-terminal to (2), or C-terminal to (2).

In certain embodiments, (1) is C-terminal to (2). In this embodiment, optionally, (1) is SEQ ID NO: 2.

In certain embodiments, (1) is N-terminal to (2). In this embodiment, optionally, (1) is SEQ ID NO: 1.

In certain embodiments, the fusion protein may comprise two or more polypeptides each comprising an amino acid sequence having at least 90% identity to SEQ ID NO: 1 or 2.

For example, in some embodiments, the fusion protein may comprise both SEQ ID NOs: 1 and 2. In this embodiment, the heterologous polypeptide may be flanked by two polypeptides, with an N-terminal polypeptide comprising an amino acid sequence having at least 90% identity to SEQ ID NO: 1, and a C-terminal polypeptide comprising an amino acid sequence having at least 90% identity to SEQ ID NO: 2. In another embodiment, the heterologous polypeptide may be flanked by three or more polypeptides, each comprising an amino acid sequence having at least 90% identity to SEQ ID NO: 1 or 2, wherein any polypeptides comprising an amino acid sequence having at least 90% identity to SEQ ID NO: 1 is N-terminal to the heterologous polypeptide, and/or any polypeptides comprising an amino acid sequence having at least 90% identity to SEQ ID NO: 2 is C-terminal to the heterologous polypeptide. For example, there can be two (identical or different) polypeptides each comprising an amino acid sequence having at least 90% identity to SEQ ID NO: 1, both are N-terminal to the heterologous polypeptide, and one polypeptides comprising an amino acid sequence having at least 90% identity to SEQ ID NO: 2, which is C-terminal to the heterologous polypeptide, etc.

Thus in certain embodiment, the fusion protein further comprises (3) a second polypeptide comprising an amino acid sequence having at least 90% identity to SEQ ID NO: 1 or 2, wherein (1) and (3) each comprising a different one of SEQ ID NO: 1 and 2.

In certain embodiments, the polypeptide of SEQ ID NO: 1 is fused N-terminal to the heterologous polypeptide, and the polypeptide of SEQ ID NO: 2 is fused C-terminal to the heterologous polypeptide.

In certain embodiments, the fusion protein may comprise only SEQ ID NOs: 1 or 2, but not both. For example, the fusion protein may comprise one or more (identical or different) polypeptides each comprising an amino acid sequence having at least 90% identity to SEQ ID NO: 1, and all such polypeptides are N-terminal to the heterologous polypeptide. In certain embodiments, the fusion protein may comprise one or more (identical or different) polypeptides each comprising an amino acid sequence having at least 90% identity to SEQ ID NO: 2, and all such polypeptides are C-terminal to the heterologous polypeptide.

In certain embodiments, the polypeptide comprises an amino acid sequence having at least 90%, 91%, 92%, 93%, 94%, 95%, 96%, 97%, 98% or 99% identity to SEQ ID NO: 1. The sequence percentage identity may be based on the query sequence (i.e., the polypeptide different from SEQ ID NO: 1), SEQ ID NO: 1, or the aligned sequence of the query and SEQ ID NO: 1.

In certain embodiments, the polypeptide comprises an amino acid sequence having at least 90%, 91%, 92%, 93%, 94%, 95%, 96%, 97%, 98% or 99% identity to SEQ ID NO: 2. The sequence percentage identity may be based on the query sequence (i.e., the polypeptide different from SEQ ID NO: 2), SEQ ID NO: 2, or the aligned sequence of the query and SEQ ID NO: 2.

In certain embodiments, the heterologous polypeptide is a polypeptide in need to extension serum half-life in an animal, such as in human or a non-human mammal. In certain embodiments, the heterologous polypeptide is a polypeptide having relatively short serum half-life (e.g., about 10 min., 15 min., 20 min., 30 min., 45 min., 1 hr, or 2 hrs).

In certain embodiments, the heterologous polypeptide is a therapeutic polypeptide.

As used herein, “therapeutic polypeptide” includes polypeptides being the subject of pharmaceutical research and development (R&D) for human and/or veterinarian use in treating a disease or indication. In certain embodiments, therapeutic polypeptide refers to peptide or protein therapeutics are currently being or having been evaluated in clinical trials.

The list of therapeutic polypeptides that have been or are currently the subject of pharmaceutical R&D and/or clinical development can be obtained from public or proprietary sources. For example, the Peptide Therapeutics Foundation (PTF) maintained and made publicly available a dataset of commercially sponsored protein therapeutics that had entered clinical study. Additional data can be collected from the public sources, e.g., clinicaltrials.gov, PubMed, company and regulatory agency websites, etc.; as well as proprietary or commercial databases (e.g. Thomson Reuters Partnering, Thomson Reuters Integrity, Sagient Research Systems BioMedTracker etc.).

In certain embodiments, therapeutic polypeptide include peptides with a single polypeptide chain, such as those with a length of no more than 500 amino acids, 450 amino acids, 400 amino acids, 350 amino acids, 300 amino acids, 250 amino acids, 200 amino acids, 150 amino acids, 100 amino acids, 80 amino acids, 50 amino acids, 40 amino acids, 30 amino acids, or 20 amino acids. In certain embodiments, therapeutic polypeptide includes two or more polypeptides with linked or associated together via one or more disulfide bond(s), such as those with a combined length of no more than 1500 amino acids, 1000 amino acids, 800 amino acids, 700 amino acids, 600 amino acids, 500 amino acids, 450 amino acids, 400 amino acids, 350 amino acids, 300 amino acids, 250 amino acids, 200 amino acids, 150 amino acids, 100 amino acids, 80 amino acids, or 50 amino acids.

In certain embodiments, therapeutic polypeptide includes peptides with a single polypeptide chain, such as those with a length of no more than 500 amino acids, 450 amino acids, 400 amino acids, 350 amino acids, 300 amino acids, 250 amino acids, 200 amino acids, 150 amino acids, 100 amino acids, 80 amino acids, 50 amino acids, 40 amino acids, 30 amino acids, or 20 amino acids.

In certain embodiments, exemplary but non-limiting heterologous polypeptide includes fibroblast growth factor 21 (FGF21), follicle-stimulating hormone (FSH), myeloid-derived growth factor (MYDGF), fibroblast growth factor binding protein 3 (FGFBP3), natriuretic peptides B, cholecystokinin, glucagon-like peptide-1 (GLP-1), gonadotropin-releasing hormone, secretin, leuprorelin, enfuvirtide, glucagon, bivalirudin, sermorelin, corticotropin tetracosapeptide, insulin-like growth factor (IGF), parathyroid hormone, or amylin.

In certain embodiments, the therapeutic polypeptide has a (human or mouse) serum half-life that is at least 10%, 20%, 30%, 40%, 50%, 60%, 70%, 80%, 85%, 90%, or 95% less than that of the fusion protein.

In certain embodiments, the fusion protein comprises an O- and/or an N-linked glycosylation.

In certain embodiments, the fusion protein comprises sialylation.

In certain embodiments, the fusion protein further comprises a linker peptide between (1) (the polypeptide comprising an amino acid sequence having at least 90% identity to SEQ ID NO: 1 or 2) and (2) (the heterologous polypeptide).

In certain embodiments, in the fusion protein, (1) is C-terminal to (2) and is optionally SEQ ID NO: 2, and the heterologous polypeptide is MYDGF or a functional fragment thereof.

In certain embodiments, the fusion protein has an amino acid sequence having at least 90% identity to SEQ ID NO: 3, such as SEQ ID NO: 3.

Another aspect of the invention also provides a polynucleotide encoding any one of the fusion protein of the invention.

In certain embodiments, the polynucleotide is codon-optimized for expression in a target host cell.

In certain embodiments, the target host cell is a human cell, a rodent cell (e.g., a mouse cell), or a non-human mammalian cell.

Another aspect of the invention provides a vector comprising the polynucleotide of the invention.

In certain embodiments, the vector is an expression vector, such as a plasmid.

Another aspect of the invention provides a host cell comprising the fusion protein of the invention, the polynucleotide of the invention, or the vector of the invention.

In certain embodiments, the host cell is a tissue culture cell.

In certain embodiments, the host cell is a CHO cell or a HEK293 cell or derivative thereof.

Another aspect of the invention provides a method of enhancing serum half-life for a protein, comprising fusing the protein to a polypeptide comprising an amino acid sequence having at least 90% identity to SEQ ID NO: 1 or 2.

In certain embodiments, the polypeptide consists of the amino acid sequence of SEQ ID NO: 1 or 2.

In certain embodiments, the protein is fused N-terminal to SEQ ID NO: 2.

In certain embodiments, the protein is fused to the polypeptide via a linker polypeptide.

Another aspect of the invention provides a method of treating a disease, disorder, or condition in a subject in need thereof, the method comprises administering to the subject a therapeutically effective amount of the fusion protein of the invention, the polynucleotide of the invention, or the vector of the invention, wherein the disease, disorder, or condition is treatable by said heterologous polypeptide.

In certain embodiments, the disease, disorder, or condition is selected from the group consisting of a tissue injury, a cardiovascular disease, an inflammatory disease or disorder, and a kidney disease.

In certain embodiments, the tissue injury is an acute injury such as myocardio infarction or stroke.

In certain embodiments, the tissue injury is a chronic injury such as diabetic injury to kidney.

In certain embodiments, the cardiovascular disease is selected from the group consisting of myocardial infarction, arteriosclerosis, hypertension, angina pectoris, hyperlipidemia, and heart failure.

In certain embodiments, the inflammatory disease or disorder is selected form the group consisting of Type I diabetes, Type II diabetes, pancreatitis, nonalcoholic fatty liver disease (NAFLD), and nonalcoholic steatohepatitis (NASH).

In certain embodiments, the disease or disorder is a kidney disease.

In certain embodiments, the subject is a human.

With the general aspects of the invention briefly described herein, specific embodiments in certain aspects of the invention are provided hereinbelow. It should be understood that any one embodiment, including those only disclosed in the examples, claims, or one section of the specification, can be combined with any one or more other embodiments of the invention unless expressly disclaimed or otherwise improper.

### 2. CD164

CD164 is also known as sialomucin or endolyn. Its 197-residue human isoform 1 precursor sequence is RefSeq NP_006007, including the N-terminal 23-residue signal peptide (bold), the mucin domains I and II (SEQ ID NOs: 1 and 2, both double underlined):

MSRLSRSLLWAATCLGVLCVLSADKNTTQHPNVTTLAPIS
NVTSAPVTSLPLVTTPAPETCEGRNSCVSCFNVSVVNTTC
FWIECKDESYCSHNSTVSDCQVGNTTDFCSVSTATPVPTA
NSTAKPTVQPSPSTTSKTVTTSGTTNNTVTPTSQPVRKST
FDAASFIGGIVLVLGVQAVIFFLYKFCKSKERNYHTL

Using the above human sequence without the signal peptide as a query, BLASTp search in the NCBI nr database retrieved numerous homologs (including orthologs and paralogs) from other species. The least homologous primate homolog from Aotus nancymaae shares 88% sequence identity to the query, and the least homologous homolog in the top 100 hits is a rodent (Castor canadensis) sharing 69% sequence homology.

Similarly, using SEQ ID NO: 2 as the query, BLASTp search identified 99 hits, with higher primate homologs generally sharing >98% sequence identity, lower primate (such as Mandrillus leucophaeus) homologs generally sharing about 90-94% sequence identity. Rodents such as rats and mice generally share about 70% sequence identity in this region.

Thus in certain embodiments, the fusion protein of the invention includes a mammalian CD164 mucin domain I or H sharing at least about 70%, 80%, 90%, 95%, 97%, or 99% sequence identity with the human CD164 mucin domain I or H (SEQ ID NOs: 1 or 2, respectively).

In certain embodiments, the mammalian CD164 mucin domain I or II is SEQ ID NO: 1 or 2.

In certain embodiments, the fusion protein of the invention comprises a variant, mutant, or synthetic CD164 mucin domain I or II sharing at least about 90%, 95%, 97%, or 99% sequence identity with the human CD164 mucin domain I or II (SEQ ID NOs: 1 or 2, respectively). In certain embodiments, the variant, mutant, or synthetic CD164 mucin domain I or II has identical N- and/or O-glycosylation sites as wild-type human CD164 mucin domains I or II, respectively.

### 3. Therapeutic Polypeptides and Treatable Diseases

In certain embodiments, the fusion protein of the invention comprises a heterologous polypeptide that is a therapeutic polypeptide, such as any one disclosed in Kaspar and Reichert, “Future directions for peptide therapeutics development,” Drug Discov. Today, 18:807-817, 2013; and Fosgerau and Hoffmann, Drug Discov. Today, 20(1):122-128, 2015 (both incorporated herein by reference).

Naturally occurring peptides are often not directly suitable for use as convenient therapeutics because they have intrinsic weaknesses, including poor chemical and physical stability, and a short circulating plasma half-life. These aspects must be addressed for their use as medicines.

Therapeutic polypeptides typically also have short half-lives (i.e. minutes) in circulation, which severely limits therapeutic utility, and requires frequent dosing (usually via IV or other injection that either requires a hospital trip or stay or trained patient self-administration). Thus being able to pro-long drug half-life in circulation is a major improvement for patient convenience, compliance, cost, and ultimately therapeutic efficacy.

As of 2015, there are more than 60 US Food and Drug Administration (FDA)-approved peptide medicines on the market, with approximately 140 peptide drugs in clinical trials and more than 500 therapeutic peptides in preclinical development. These numbers have grown significantly since. Thus the fusion protein of the invention provides a general approach to improve the therapeutic half-life of existing and developing therapeutic polypeptides, including those already approved by FDA and EMA.

In certain embodiments, the therapeutic polypeptide is useful to treat a metabolic disease, a cancer, an inflammation, or as a vaccine.

In certain embodiments, the therapeutic polypeptide is useful to treat an endocrinological disease, a respiratory disease, a bone disease, a urological disease, an ophthamological disease, a dermatological disease, a CNS disease, pain, a gastroenterologicla disease, allergy/immunological disease, an infectious disease, a cardiovescular disease, an oncological disease, or a metabolic disease.

In certain embodiments, the therapeutic polypeptide is useful to treat gastrointestinal disorders such as short bowel syndrome (e.g., linaclotide and teduglutide).

In certain embodiments, the therapeutic polypeptide is useful to treat respiratory distress syndrome, such as one in high-risk, premature infants e.g., lucinactant).

In certain embodiments, the therapeutic polypeptide is useful to treat anemia, such as anemia in adult dialysis patients who have chronic kidney disease (e.g., peginesatide).

In certain embodiments, the therapeutic polypeptide is useful to treat Cushing's disease, such as Cushing's disease in adult patients for whom pituitary surgery is not an option or has not been curative (e.g., pasireotide).

In certain embodiments, the therapeutic polypeptide is useful to treat cancer, such as a hematological cancer (e.g., carfilzomib).

In certain embodiments, the therapeutic polypeptide is useful to treat erythropoietic protoporphyria (EPP), a rare genetic disease characterized by severe reactions to sunlight (e.g., the photoprotectant afamelanotide, a melanocortin 1 receptor agonist).

In certain embodiments, the therapeutic polypeptide is useful to treat EPP and solar urticaria (e.g., afamelanotide).

In certain embodiments, the therapeutic polypeptide is useful to treat multiple sclerosis, such as glatiramer acetate.

In certain embodiments, the therapeutic polypeptide is euprolide, octreotide (treatment for acromegaly and symptoms in cancer patients), or goserelin (management of endometriosis and palliative treatment of advanced prostate and breast cancer).

In certain embodiments, the therapeutic polypeptide is an antibody or antigen-binding fragment thereof, such as scFv, Fab, Fab′, F(ab′)₂, Fd, disulfide linked Fv, V-NAR domain, IgNar, intrabody, IgGΔCH2, minibody, F(ab′)₃, tetrabody, triabody, diabody, single-domain antibody, DVD-Ig, Fcab, mAb₂, (scFv)₂, or scFv-Fc.

In certain embodiments, the therapeutic polypeptide is a natural polypeptide, such as protein fragments, degradation products, or signaling molecules originating from the gut microbiome.

In certain embodiments, the therapeutic polypeptide is an antibody-drug conjugates (ADCs; such as gemtuzumab-ozogamicin, brentuximab-vedotin, trastuzumab-emtansine).

In certain embodiments, the therapeutic polypeptide is a peptide-drug conjugate (PDC; such as zoptarelin doxorubicin, EP100), for treating urothelial carcinoma, endometrial cancer, prostate cancer, breast cancer, and ovarian cancer.

In certain embodiments, the therapeutic polypeptide targets a GLP receptor, a CXCR4, an opioid receptor, a Ghrelin receptor, a GNRH-R, a vasopressin, an oxytocin receptor, a melanocortin receptor, or a parathyroid hormone receptor.

In certain embodiments, the therapeutic polypeptide is useful to treat type 2 diabetes or obesity, such as a GLP-1R agonist polypeptide (e.g., lixisenatide; exenatide/Byetta1/Bydureon1; liraglutide; Albiglutide (an albumin fusion); Dulaglutide (Fc fusion); semaglutide (acylated GLP-1 analog); PB1023 (recombinant GLP-1 analog fused to a biopolymer; Cpd86; ZPGG-72; ZP3022; MOD-6030; ZP2929; HM12525A; VSR859; NN9926; TTP273/TTP054; ZYOG1; MAR709; TT401; HM11260C; ITCA); R06811135; ZP2929; TT401). These therapeutic polypeptides can also be used to treat cardiovascular diseases, neurodegenerative disorders and for weight management, such as myocardial infarction, Alzheimer's disease, Parkinson's disease, and mild cognitive impairment.

In certain embodiments, the therapeutic polypeptide is a multifunctional peptide such as GLP-1-GIP and GLP-1-GCG dual agonist. For example, the GLP-1-GCG dual agonist provides a greater weight loss in overweight patients with T2DM compared with a pure GLP-1 agonist, via a GCG-derived increase in energy expenditure. Meanwhile, the GLP-1-CCKB dual agonist, in which the CCKB (gastrin) agonism is added to the GLP-1 action, enhances the pancreatic β cell function, which in turn aid in minimizing/preventing T2DM progression.

The single most frequent target for peptide therapeutics evaluated in clinical studies is GLP-1R. Of the 265 peptide therapeutics that entered clinical study during 2000-2012, 32 (12.1%) were GLP-1R agonists. Meanwhile, all other targets were below 3% in frequency. GLP-1R is well-validated as a target for type 2 diabetes drugs. Since the approval of exenatide in 2005, a notable trend in this product category has been the development of peptides designed, formulated or delivered such that they can be dosed less frequently than the twice-daily regimen of exenatide. The endogenous ligand GLP-1 is degraded within 1-2 min by dipeptidyl peptidase 4 (DPP4). Exenatide, which has a half-life of about 2.4 hours, and lixisenatide (which also has a half-life of 2-4 hours), were specifically designed to be DDP4-resistant. The peptide backbone of liraglutide is modified by addition of a lipid (i.e. palmitic acid), thus increasing its half-life to 13 hours, enabling liraglutide to be administered once daily. Albiglutide comprises a tandem repeat of a DDP4-resistant GLP-1(7-36)amide analog fused to HAS. Its half-life is 6-7 days. Dulaglutide comprises a DDP4-resistant GLP-1(7-36)amide analog fused to the Fc region of an IgG4 that was engineered to reduce binding to Fcg receptors and the potential for immunogenicity, and eliminate half-antibody formation. It has a half-life of around four days. Semaglutide is an acylated GLP-1 analog with a half-life of 6-7 days.

In certain embodiments, the therapeutic polypeptide is MYDGF. Myeloid-derived growth factor (MYDGF, also known as C19orf10) is a paracrine-acting cytokines produced by bone marrow-derived monocytes and macrophages, and has been shown to be capable of promoting cardiac recovery after ischemic myocardial infarction (MI). MYDGF also maintains glucose homeostasis by inducing glucagon-like peptide-1 (GLP-1) production and secretion, leading to improved glucose tolerance and lipid metabolism. MYDGF protects podocytes from injury by preserving slit diaphragm protein expression and decreasing podocyte apoptosis in diabetic kidney disease (DKD).

Although administration of MYDGF by continuous intravenous administration or adenovirus overexpression suppresses injure of organs and tissues in animal models, the utility in clinical settings is limited due to its short half-life in blood (approximately 15.3 minutes). In order to decrease the renal filtration rate, we developed MYDGF with longer serum half-life.

Thus the subject fusion protein of MYDGF with CD164 mucin domain can be used to treat tissue injury, a cardiovascular disease, an inflammatory disease or disorder, and a kidney disease.

In certain embodiments, the therapeutic polypeptide is FGF21. Fibroblast growth factor 21 (FGF21) is an endocrine molecule belonging to the FGF superfamily, and has been show to function in metabolism maintaining lipid and energy homeostasis (Hecht 2012). FGF21 is a therapeutic in treating diabetes, pancreatitis, nonalcoholic fatty liver disease (NAFLD), and nonalcoholic steatohepatitis (NASH).

In certain embodiments, the therapeutic polypeptide is FSH. Follicle-stimulating hormone (FSH) is a gonadotropin, a glycoprotein polypeptide hormone, which is synthesized and secreted by gonadotropic cells of the anterior pituitary gland.

In certain embodiments, the therapeutic polypeptide is FGFBP3. Fibroblast growth factor binding protein 3 (FGFBP3) are secreted chaperones known to modulate fat and glucose metabolism. FGFBP3 is a potential therapeutic for the treatment of nonalcoholic fatty liver disease and type 2 diabetes mellitus (Tassi et al. 2018).

It should be noted that the above described therapeutic polypeptides are merely for illustrative purpose only, and numerous other therapeutic polypeptides, especially those formulated for IV injection but with a relatively short circulation half-life, are within the scope of the present invention.

### 4. Polynucleotide and Vectors

Another aspect of the invention provides a polynucleotide encoding the fusion protein of the invention described herein. In one embodiment, the polynucleotide encodes any one of SEQ ID NOs: 1-8, such as any one of SEQ ID NOs: 3-8.

In some embodiments, the polynucleotide is a synthetic nucleic acid. In some embodiments, the polynucleotide is a DNA molecule. In some embodiments, the polynucleotide is an RNA molecule (e.g., an mRNA molecule). In some embodiments, the mRNA is capped, polyadenylated, substituted with 5-methyl cytidine, substituted with pseudouridine, or a combination thereof.

In some embodiments, the polynucleotide (e.g., DNA) is operably linked to a regulatory element (e.g., a promoter) in order to control the expression of the polynucleotide. In some embodiments, the promoter is a constitutive promoter. In some embodiments, the promoter is an inducible promoter. In some embodiments, the promoter is a cell-specific promoter. In some embodiments, the promoter is an organism-specific promoter.

Suitable promoters are known in the art and include, for example, a pol I promoter, a pol II promoter, a pol M promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, and a β-actin promoter.

In one aspect, the present disclosure provides polynucleotide sequences that are at least 50%, 55%, 60%, 65%, 70%, 75%, 80%, 85%, 90%, 91%, 92%, 93%, 94%, 95%, 96%, 97%, 98%, 99%, or 100% identical to the polynucleotide sequences described herein, i.e., nucleic acid sequences encoding any of the fusions described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

In certain embodiments, the nucleic acid molecule encoding the fusion proteins, derivatives or functional fragments thereof are codon-optimized for expression in a host cell or organism. The host cell may include established cell lines or isolated primary cells. The polynucleotide can be codon optimized for use in any organism of interest, in particular human immune cells. Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference). Computer algorithms for codon optimizing a particular sequence for expression in a particular host cell are also available, such as Gene Forge (Aptagen; Jacobus, Pa.).

An example of a codon optimized sequence, is in this instance a polynucleotide coding sequence optimized for expression in a eukaryote, e.g., humans (i.e. being optimized for expression in humans), or for another eukaryote, animal or mammal as herein discussed). Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at http://www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a fusion correspond to the most frequently used codon for a particular residue.

In some embodiments, the polynucleotide(s) or nucleic acid(s) of the invention are present in a vector (e.g., a viral vector).

The term “vector” as used herein generally refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

In certain embodiments, the vector can be a cloning vector, or an expression vector. The vectors can be plasmids, phagemids, Cosmids, etc. The vectors may include one or more regulatory elements that allow for the propagation of the vector in a cell of interest (e.g., a mammalian cell such as a CHO cell, HEK293 cell, etc).

In certain embodiments, the vector is a “plasmid,” which refers to a circular double stranded DNA loop into which additional DNA segments can be inserted, such as by standard molecular cloning techniques.

In certain embodiments, the vector is a viral vector, wherein virally-derived DNA or RNA sequences are present in the vector for packaging into a virus (e.g., retroviruses, lentiviruses, replication defective retroviruses, adenoviruses, replication defective adenoviruses, HSV, and adeno-associated viruses (AAV)). Viral vectors also include polynucleotides carried by a virus for transfection into a host cell.

In certain embodiments, the vector is a lentiviral vector. In certain embodiments, the lentiviral vector is a self-inactivating lentiviral vector. See, for example, Zufferey et al., “Self-Inactivating Lentivirus Vector for Safe and Efficient In vivo Gene Delivery.” J Virol. 72(12): 9873-9880, 1998 (incorporated herein by reference).

In certain embodiments, the vector is based on the Sleeping Beauty (SB) transposon, which has been used as a non-viral vector for introducing genes into genomes of vertebrate animals and for gene therapy. Because the SB system is composed solely of DNA, the costs of production and delivery are considerably reduced compared to viral vectors. SB transposons have been used to genetically modify T cell in human clinical trials.

In certain embodiments, the vector is capable of autonomous replication in a host cell into which they are introduced. In certain embodiments, the vector (e.g., non-episomal mammalian vectors) is integrated into the genome of a host cell upon introduction into the host cell, and thereby are replicated along with the host genome. In certain embodiments, the vector, referred to herein as “expression vector,” is capable of directing the expression of genes to which they are operatively-linked. Vectors for and that result in expression in a eukaryotic cell are “eukaryotic expression vectors.”

In certain embodiments, the vector is a recombinant expression vector that comprises a nucleic acid of the invention in a form suitable for expression of the nucleic acid in a host cell. The recombinant expression vector may include one or more regulatory elements, which may be selected on the basis of the host cells to be used for expression, that is operatively-linked to the nucleic acid sequence to be expressed. Here, “operably linked” means that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes such as T cells, or NK cells). Regulatory elements may also direct expression in a temporal-dependent manner, such as in a cell-cycle dependent or developmental stage-dependent manner, which may or may not also be tissue or cell-type specific.

In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and Hl promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 1-actin promoter, the phosphoglycerol kinase (PGK) promoter, and the EF1a promoter.

Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer: and the intron sequence between exons 2 and 3 of rabbit b-globin (Proc. Natl. Acad. Sci. USA., Vol. 78(3), p. 1527-31, 1981).

It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein.

In certain embodiments, the vector is a lentiviral or AAV vector, which can be selected for targeting particular types of cells (e.g., with tissue and/or cell type-specific tropism).

The vectors of the invention can be introduced into a target or host cell, using any of many art-recognized methods, such as transfection, lipid vectors, infection, electroporation, microinjection, parenteral injections, aerosol, gene guns, or use of ballistic particles, etc.

In certain embodiments, the fusion proteins described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO—S and DG44 cells; PERC6@D cells (Crucell); and NSO cells. In some embodiments, fusion protein described herein may be expressed in yeast. See, e.g., U.S. Publication No. US 2006/0270045 A1. In some embodiments, a particular eukaryotic host cell is selected based on its ability to make desired post-translational modifications of the mucin domains. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc., Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more vectors or polynucleotides of the invention encoding the subject fusion protein, according to any suitable method.

In certain embodiments, transfection includes chemical transfection that introduces the vector by, e.g., calcium phosphate, lipid, or protein complexes. Calcium phosphate, DEAE-dextran, liposomes, and lipoplexes (for oral delivery of gene) surfactants and perfluro chemical liquids for aerosol delivery of gene.

In certain embodiments, lipid vectors are generated by a combination of plasmid DNA and a lipid solution that result in the formation of a liposome, which can be fused with the cell membranes of a variety of cell types, thus introducing the vector DNA into the cytoplasm and nucleus, where the encoded gene is expressed. In certain embodiments, folate is linked to DNA or DNA-lipid complexes to more efficiently introduce vectors into cells expressing high levels of folate receptor. Other targeting moieties can be similarly used to target the delivery of the vectors to specific cell types targeted by the targeting moieties.

In certain embodiments, the vector DNA is internalized via receptor-mediated endocytosis.

In certain embodiments, the vector is a lentiviral vector, and the target cell infection spectrum of the vector is expanded by replacing the genes for surface glycoproteins with genes from another viral genome in the packaging cell lines packaging cell lines (PCL) of the vector.

### 5. Pharmaceutical Composition

Another aspect of the present invention provides a pharmaceutical composition for the treatment of a disease or condition, such as cancer or inflammatory disease, or any other disease or indication treatable by the therapeutic polypeptides described herein. The pharmaceutical composition comprises a therapeutically effective amount of the fusion protein of the invention, the polynucleotide of the invention, or the vector of the invention. The pharmaceutical composition further comprises a pharmaceutically acceptable carrier or excipient.

As used herein, “pharmaceutically acceptable carrier or excipient” includes any and all solvents, dispersion media, coatings, antibacterial and antifungal agents, isotonic and absorption delaying agents, and the like physiologically compatible. In certain embodiments, the carrier is suitable for intravenous, intramuscular, subcutaneous, parenteral, spinal or epidermal administration (e.g., by injection or infusion). In certain embodiments, the pharmaceutical composition comprising the fusion protein of the invention is formulated with a carrier or excipient for administration intravenously (i.v.), subcutaneously (s.c.), inhalation, or orally (e.g., oral delivery of peptides directly expressed in the gastrointestinal tract).

In other embodiment, the pharmaceutical composition comprising the fusion protein of the invention is formulated with a carrier or excipient for intranasal, transdermal, transbuccal (e.g., delivery via the combination of gold nanoparticles (Midatech) and the PharmFilm™ (Monosol Rx) technology) administration.

As used herein, a “therapeutically effective amount” or “therapeutically effective dose” or “effective amount” means administering a sufficient amount of a substance, compound, material or cell to produce a desired therapeutic effect. Therefore, the administered amount is sufficient to prevent, cure, or ameliorate at least one symptom of, or completely or partially blocking the progression/worsening of the disease or condition. The administered amount is also below a threshold toxicity level, above which could/would cause the subject to terminate or discontinue with the therapy.

The amount and the dosage level of the fusion protein in the pharmaceutical composition of the invention may be varied depending on specific patient need, the mode of administration, the type and/or degree of disease in a subject, the desired therapeutic response, the tolerable toxicity to the patient, as well as other factors deemed relevant by an attending physician. That is, the selected dosage level may depend on a variety of pharmacokinetic factors including the particular composition used, the route of administration, the age of the patient, other pharmaceutical composition used in conjunction, duration and time of administration, rate of excretion or elimination, gender, weight, condition, general health condition and medical history, and like factors of the patient, as is generally known in the medical field. One of ordinary skill in the art can empirically determine the effective amount of the invention without necessitating undue experimentation. Combined with the teachings provided herein, by choosing among the various fusion proteins of the invention, and weighing factors such as potency, relative bioavailability, patient body weight, severity of adverse side-effects and preferred mode of administration, an effective prophylactic or therapeutic treatment regimen can be planned which does not cause substantial toxicity in and of itself and yet is entirely effective to treat the particular subject.

Toxicity and efficacy of the protocols of the present invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

In certain embodiments, data obtained from the cell culture assays, animal studies and human studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

In certain embodiments, the pharmaceutical composition is formulated for use in a subject such as a human, non-human primate, cow, horse, pig, sheep, goat, dog, cat, or rodent. In certain embodiments, the subject is a human subject.

### EXAMPLES

### Example 1 CD164 Mucin Domain Fusion Proteins and Characterization Methods

Genes containing the CD164 mucin domain I (aa 24-60) and mucin domain II (aa 110-162) were codon-optimized for recombinant expression. Typically, the mucin domain I was fused to N-termini of selected proteins, while the mucin domain II was fused to C-termini.

At least the following fusion proteins were constructed: fibroblast growth factor 21 (FGF21), follicle-stimulating hormone (FSH), myeloid-derived growth factor (MYDGF), and Fibroblast Growth Factor Binding Protein 3 (FGFBP3), all of which are current or potential protein therapeutics. The sequences of the mucin domains and the several representative fusions thereof are provided below:

CD164 mucin domain I:
(SEQ ID NO: 1)
DKNTTQHPNVTTLAPISNVTSAPVTSLPLVTTPAPET.
CD164 mucin domain II:
(SEQ ID NO: 2)
SVSTATPVPTANSTAKPTVQPSPSTTSKTVTTSGTTNNTVTPTSQPVRKS
TFD.
MYDGF-164 fusion:
(SEQ ID NO: 3)
VSEPTTVAFDVRPGGVVHSFSHNVGPGDKYTCMFTYASQGGTNEQWQMSL
GTSEDHQHFTCTIWRPQGKSYLYFTQFKAEVRGAEIEYAMAYSKAAFERE
SDVPLKTEEFEVTKTAVAHRPGAFKAELSKLVIVAKASRTELSVSTATPV
PTANSTAKPTVQPSPSTTSKTVTTSGTTNNTVTPTSQPVRKSTFD
CD164-MYDGF fusion:
(SEQ ID NO: 4)
DKNTTQHPNVTTLAPISNVTSAPVTSLPLVTTPAPETVSEPTTVAFDVRP
GGVVHSFSHNVGPGDKYTCMFTYASQGGTNEQWQMSLGTSEDHQHFTCTI
WRPQGKSYLYFTQFKAEVRGAEIEYAMAYSKAAFERESDVPLKTEEFEVT
KTAVAHRPGAFKAELSKLVIVAKASRTE
FGF21-164 fusion:
(SEQ ID NO: 5)
DSSPLLQFGGQVRQRYLYTDDAQQTEAHLEIREDGTVGGAADQSPESLLQ
LKALKPGVIQILGVKTSRFLCQRPDGALYGSLHFDPEACSFRELLLEDGY
NVYQSEAHGLPLHCPGNKSPHRDPAPRGPCRFLPLPGLPPALPEPPGILA
PQPPDVGSSDPLSMVGPSQGRSPSYASSVSTATPVPTANSTAKPTVQPSP
STTSKTVTTSGTTNNTVTPTSQPVRKSTED

In certain embodiments, FGF21 in 164 fusion is a wild-type FGF21 sequence. In certain embodiments, FGF21 in 164 comprises an L146C and/or an A162C mutation.

FGFBP3-164 fusion:
(SEQ ID NO: 6)
RREKGAASNVAEPVPGPTGGSSGRFLSPEQHACSWQLLLPAPEAAAGSEL
ALRCQSPDGARHQCAYRGHPERCAAYAARRAHFWKQVLGGLRKKRRPCHD
PAPLQARLCAGKKGHGAELRLVPRASPPARPTVAGFAGESKPRARNRGRT
RERASGPAAGTPPPQSAPPKENPSERKTNEGKRKAALVPNEERPMGTGPD
PDGLDGNAELTETYCAEKWHSLCNFFVNFWNGSVSTATPVPTANSTAKPT
VQPSPSTTSKTVTTSGTTNNTVTPTSQPVRKSTFD
164-FSHa fusion:
(SEQ ID NO: 7)
DKNTTQHPNVTTLAPISNVTSAPVTSLPLVTTPAPETAPDVQDCPECTLQ
ENPFFSQPGAPILQCMGCCFSRAYPTPLRSKKTMVTSESTCCVAKSYNVA
KSYNRVTVMGGFKVENHTACHCSTCYYHKS
FSHb-164 fusion:
(SEQ ID NO: 8)
MKTLQFFFLFCCWKAICCNSCELTNITIAIEKEECRFCISINTTWCAGYC
YTRDLVYKDPARPKIQKTCTFKELVYETVRVPGCAHHADSLYTYPVATQC
HCGKCDSDSTDCTVRGLGPSYCSFGEMKESVSTATPVPTANSTAKPTVQP
SPSTTSKTVTTSGTTNNTVTPTSQPVRKSTFD

All gene fragments were cloned into pD2531 expression vector (ATUM), and recombinant expression of the fusion proteins were carried out by stable transfection of Chinese Hamster Ovary (CHO) or 293F cells that were previously adapted to serum-free growth. Recombinant cell lines were selected in growth media lacking glutamine, and subcloned in 96-well tissue culture plates by limiting dilutions. Recombinant protein expression was characterized initially by Western blot analysis using polyclonal antibodies (SinoBiologicals or Abcam), followed by protein purification and analytical characterization.

Protein purities were determined by SDS-PAGE with the Mini-PROTEAN® Tetra System (Bio-Rad), followed by densitometry after Coomassie Brilliant Blue (CBB) staining. The molecular weight standard (11-180 kDa, Tanon) was applied for analysis.

For purity analysis, about 10 μg of fusion proteins were solubilized in sample buffer and resolved in 12% SDS-PAGE gel, and densitometry was performed using Tanon 4600SF gel image analysis system.

Isoelectric focusing (IEF) were performed with the Multiphor II Electrophoresis System (GE Healthcare) using polyacrylamide gel electrophoresis (PAGE). The pH range 3-10 IEF Standard (Bio-Rad) and the pH range 2.5-6.5 IEF Standard (GE Healthcare) were applied to reference the pI range.

Samples were desalted prior to electro-focusing, and 10 μg protein samples were electro-focused at 15° C. by an initial focusing step at 700 V for 20 min, followed by a linear gradient to 500 V for 20 min and a final linear gradient to 2000 V for 90 min. After electro-focusing, proteins in PAGE gels were visualized by silver staining. The gel image was scanned, and pI values were determined by analysis using IMAGEQUANT TL version 7.0 (GE Healthcare) software.

Analytical SEC was performed on an AdvanceBio SEC 300 Å column (Agilent Technologies) at a flow rate of 0.3 ml/min using HPLC (High Performance Liquid Chromtography System, Agilent Technologies) equipped with an autosampler. Standard proteins (Agilent Technologies) were applied with 150 mmol/L sodium phosphate buffer (pH 7.0). The fusion proteins were applied with 150 mmol/L sodium phosphate buffer (pH 7.0). Reverse phase HPLC were performed on a ZORBAX SB300 C8 column (Agilent Technologies) at a flow rate of 1 mL/min using an elution gradient from 0% v/v acetonitrile, 0.01% v/v TFA to 60% v/v acetonitrile, 0.085% TFA.

Monosaccharides were labeled by 2-AA after hydrolyzed with 1 mL 4M trifluoroacetic acid (TFA) according to the manufacturer's protocol (Sigma). The analysis was carried out one Shimadzu Nexera UPLC system equipped with a RF-20Axs fluorescence detector and a reverse phase column (Phenomenex Hyperclone 5 μm ODS 120 Å, 250×4.60 mm). According to the monosaccharide standards and area normalization method, the composition and proportion of various monosaccharides were determined.

O-glycans and N-glycans were released from samples using an improved β-elimination. Proteins and salts were removed using a graphitized carbon cartridge (Supelclean™ ENVI™-Carb SPE) that was equilibrated with water. Glycans were eluted with 20% and 40% acetonitrile. The eluents were evaporated by reduced pressure and fluorescence labeled with 2-aminobenzamide (2-AB) and labeled glycans were separated on HILIC UPLC (ACQUITY UPLC Glycan BEH Amide Column, 130 Å, 1.7 μm, 2.1 mm×150 mm).

N-linked glycans were labeled with 2-aminobenzamide after PNGase F treatment according to the manufacturer's protocol (Sigma). Labeled glycans were separated on HILIC UPLC (ACQUITY UPLC Glycan BEH Amide Column, 130 Å, 1.7 μm, 2.1 mm×150 mm) to determine the forms of the N-linked glycans based on comparison to the 2-AB labeled dextran glycan standards.

HPLC was used to analyze the sialic acid in MYDGF-164 fusion protein. Sialic acids released from the hydrolysis of MYDGF-164 fusion protein were labeled with OPD and analyzed by UPLC-FLD or UPLC-FLD-MS (Shimadzu LCMS 2020 ESI mass spectrometer coupled with a fluorescence detector) using the same C18 reversed-phase column (Phenomenex Hyperclone 5 μm C18, 250×4.6 mm) to determine the type and content of sialic acid based on comparison to the OPD labeled sialic acids standards.

Six different exo/endoglycosidases were applied to remove the carbohydrate modification from the protein samples, including β-N-acetylhexosaminidase SN384, neuroaminidase AuNeu54, β-galactosidase Am0874, commercial galactosidase, fucosidases Eo0918, Eo3066 and Eo3141 and PNGase. The reaction mixture includes 38 μL of protein sample dissolved in water, 5 μL 200 mM PBS buffer (pH 6.5), 1 μL of each enzyme. The mixture was incubated at 37° C. for 16 h.

Mass spectra of the MYDGF-164 fusion proteins or removing glycans were acquired using a MALDI-TOF instrument, the Bruker Autoflex 244 Speed instrument equipped with a 1000 Hz Smartbeam-II laser. 2456-aza-2-thiothymine was used as matrix for protein ionization.

The molecular masses were derived from spectrum analysis using Bruker Flexanalysis software version 3.3.80.

For pharmacokinetic analysis, twelve C57BL/6 mice were injected with 17.5 mg/kg of MYDGF-164 protein by tail vein injection. Mice were randomly divided into three groups for retro-orbital sampling, and blood samples were collected at 0, 30, 240 min for the first group, 5, 60, 360 min for the second group, and 15,120,480 min for the third group after injection. Sera was obtained by centrifugation at 11000 rpm for 5 min in cold. Fusion proteins in sera were analyzed by LC-MS/MS, and the plasma concentrations of the fusion proteins was determined by using MassHunter software (Agilent, USA). Pharmacokinetic parameters based on a non-compartmental model were calculated by using Phoenix WinNonlin 7.0 software (Pharsight, USA). Cₘₐₓ and Tₘₐₓ were determined by LC-MS/MS measurement.

### Example 2 Glycosylation of Mucin Domain is Unaffected in Fusion Proteins

CD164 mucin domain I (SEQ ID NO: 1) and II (SEQ ID NO: 2) consist of 37 or 53 amino acids, respectively, and the molecular weight of the un-glycosylated forms were predicted to be 3.8 kDa or 5.4 kDa, respectively.

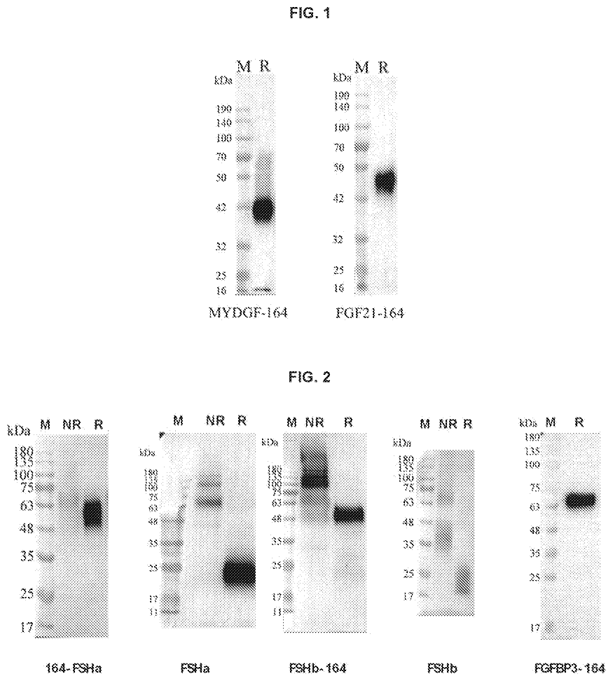

To understand whether fusion to unglycosylated proteins will impact the glycosylation of the CD164 mucin domains, chimeric proteins of these domains fused to non-glycosylated proteins MYDGF and FGF21 were constructed to create MYDGF-164 (SEQ ID NO: 3) and FGF21-164 (SEQ ID NO: 5). The fusion proteins were stably expressed in CHO or 293F cells, and secreted proteins were detected in the conditioned medium by SDS-PAGE and Western blot analysis (FIG. 1). The predicted molecular masses of mature polypeptide without glycosylation are 21.1 kDa for MYDGF-164, and 24.3 kDa for FGF21-164, respectively. SDS PAGE and Western analysis demonstrated that the MYDGF-164 migrated at 48 kDa during the electrophoresis, and FGF21-164 migrated at about 52 kDa, suggesting that the fusion to CD164 mucin domains accrued additional masses between 20 to 30 kDa.

In addition, chimeric CD164 mucin proteins to FSH alpha, FSH beta, and FGFBP3 proteins, which are all naturally glycosylated, were also generated (FIG. 2). FSH fusion proteins also gained additional masses ranging from 20 to 30 kDa, based on comparison to non-fusion proteins. Glycosylation in the CD164 mucin domains in fusion proteins proceeded in all cases tested.

These data convincingly demonstrate that glycosylation is not significantly impacted by the fusion of the mucin domains to other heterologous proteins.

### Example 3 Characterization of CD164 Mucin Fusion Proteins

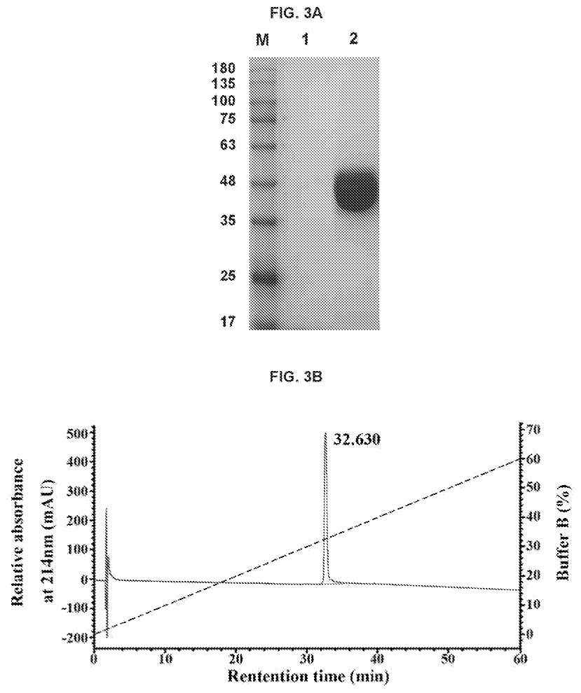

To characterize the chimeric/fusion proteins containing mucin domains, MYDGF-164 fusion was purified by conventional chromatography. The purified protein was analyzed by SDS-PAGE and CBB staining (FIG. 3A).

The purified protein migrated as 48 kDa protein, similar to that from the Western analysis (FIG. 1). The purified protein was subjected to N-terminal sequencing, and the N-terminal sequence of 5 amino acids was confirmed. The homogeneity of the protein was also confirmed by reverse phase HPLC analysis (FIG. 3B).

In RP-HPLC elution, the MYDGF-164 profile was a single absorbance peak, indicating that the two N-linked glycosylation sites of the CD164 mucin domain II were utilized in a uniform fashion, i.e., either fully utilized for glycosylation or neither was. In addition, the CBB staining of the purified protein demonstrated a relatively simple banding pattern. These observations suggested that the glycan forms of the MYDGF-164 were mostly homogeneous structure.

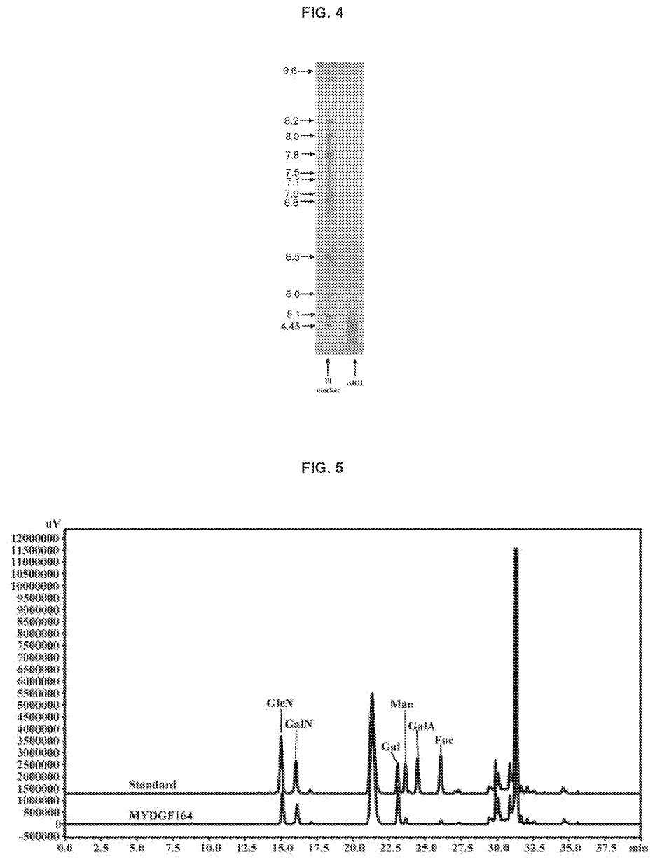

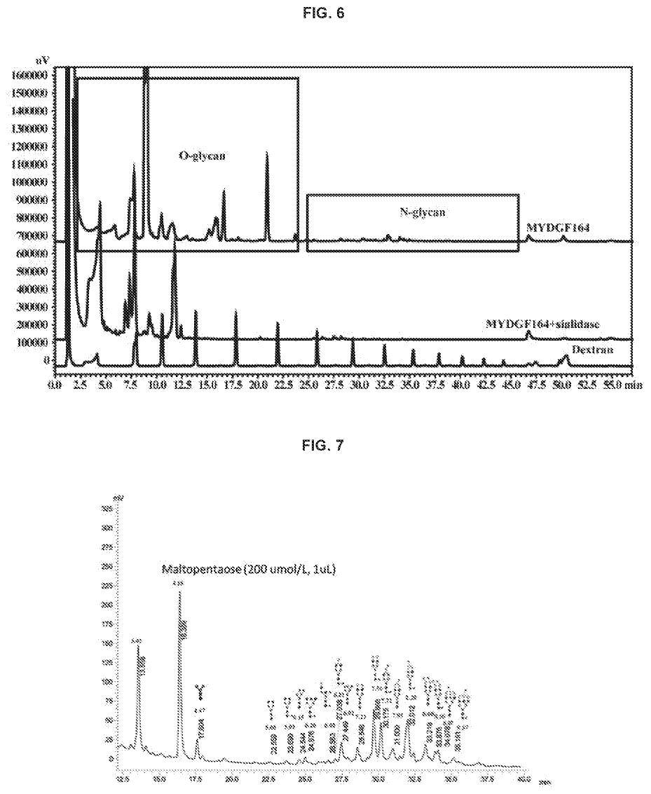

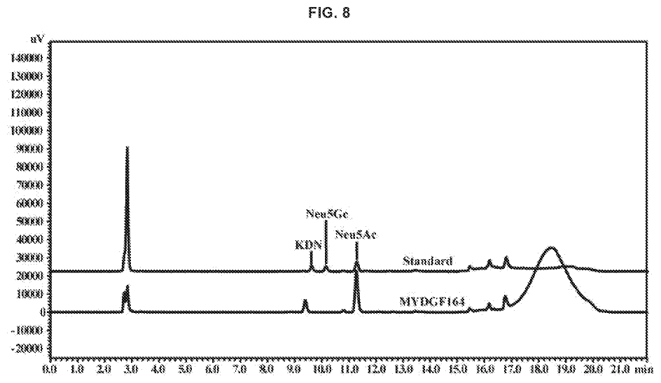

Analysis by isoelectric focusing (IEF) showed that the purified MYDGF-164 fusion were mixture components containing multiple acidic isoforms with pI 5.22-3.31 (FIG. 4). MYDGF-164 without post-translational modification is predicted to be a basic protein with a pI of 7.93. The IEF data strongly suggested that the fusion protein was converted to acidic forms, possibly by sialylation of the glycans. Sialic acids can be added to both O- and N-linked glycans.

Further analysis was conducted for N-linked glycans by PNGase treatment and 2-AB labeling, followed by analytical UPLC analysis (FIG. 7). Predominant glycan forms were biantennary, while tri-antennary glycans found at 34.078 and 35.181 minutes are negligible. Thus the relatively homogeneous biantennary glycan structures found were consistent with the CBB staining and RP-HPLC data (FIG. 3), again suggesting that the N-linked glycosylation of the CD164 mucin domain is highly homogeneous.

Therefore the presence of the N-linked glycans was confirmed. Treatment procedures for 2-AB labeling removed the sialic acids, therefore none of the elution peaks had structures with sialic acids.

For example, elution peak at 29.668 min corresponded to fucosylated bi-antennary glycan with galactoses but no sialic acid. As previously reported, the mucin II domain of CD164 contain two putative N-linked glycosylation sites (Doyonnas). Based on the analysis of the N-linked glycan, it was suggests that the MYDGF-164 fusion was fully modified at all N-glycosylation sites.

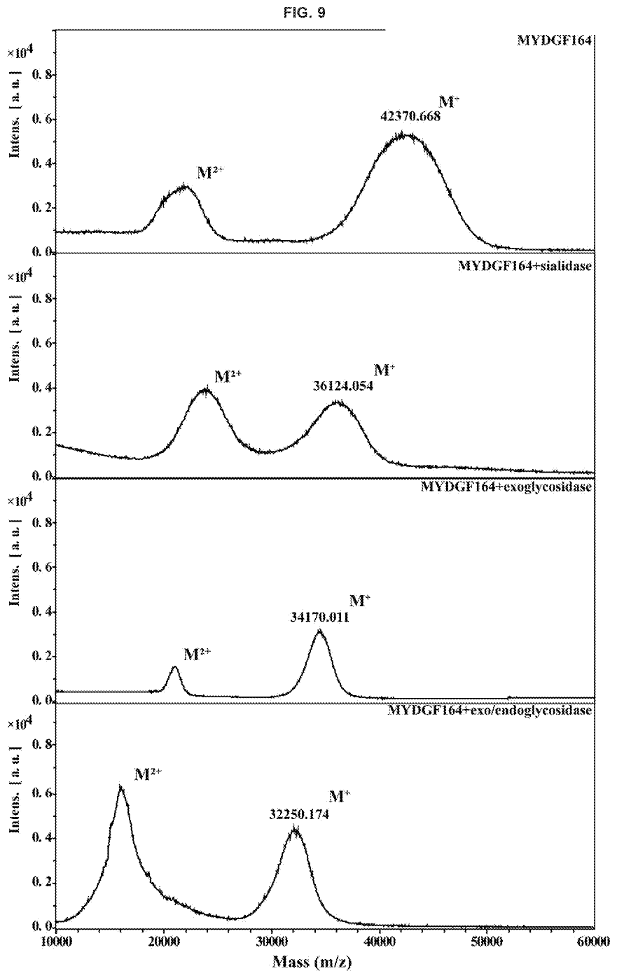

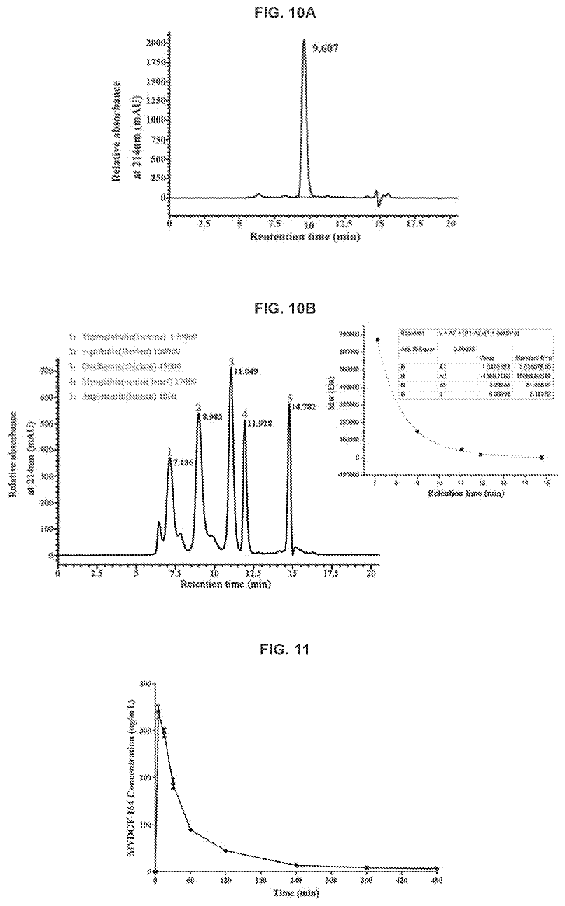

To determine the mass of the purified MYDGF-proteins, the purified protein were subjected to MALDI-TOF mass spectrometry analysis. In the mass spectrum, the purified MYDGF-164 fusion appeared as a singly charged peak at 42370.668 Dalton, and a doubly charged peak (FIG. 9). The doubly charged mass peak predicted a mass of 42,432 Dalton, which was higher compared to the mass of 42370.668 Dalton of the singly charged peak. The discrepancy is probably due to heterogeneous, laser-induced protonation of the various glyco forms of the fusion protein and subsequent release of the protonated proteins from the matrix during the MALDI-TOF. Nevertheless, compared to the predicted, 21182.6 Dalton molecular weight of the unglycosylated MYDGF-164, the mass of the singly charged peak is about 20 kDa higher, suggesting that the fusion protein is heavily glycosylated.

Hydrodynamic properties of the MYDGF-164 fusion was also characterized by analytical SEC chromatography. The protein eluted as a single, symmetric peak (FIG. 10A), corresponding to a hydrodynamic radius for a 98.744 kDa globular protein when compared to reference proteins (FIG. 10B). Due to the brush-like conformation of the mucin domain, the MYDGF-164 fusion protein unlikely folded into a globular structure. However, it is believed that, similar to Fc fusion or pegylated proteins, the increased hydrodynamic radius of the MYDGF-164 should reduce the glomerulus filtration rate, thus increase the half-life of the fusion protein in vivo.

### Example 4 Enhanced Serum Half-life of CD164 Mucin Fusion Proteins

Earlier data suggested the MYDGF has limited in vivo bioavailability due to an extremely short half-life, and its activity in a heart repair model required continuous and intravenous infusion (Korf-Klingebiel 2015).

This experiment utilized a mouse model to determine whether the half-life of the MYDGF-164 fusion was extended.

The pharmacokinetic study was carried out by intravenous injection of the purified protein followed by LC-MS/MS analysis, and concentration of the MYDGF fusion proteins in sera at various time points were quantified (FIG. 11). Pharmacokinetic parameters for the MYDGF-164 fusion protein were calculated based on a non-compartmental model (Table 1).

TABLE 1
Summary of PK data of MYDGF-164

| t_{1/2} | Tₘₐₓ | Cₘₐₓ | AUC₀₋ₜ | AUC_{0-∞} | Vₛₛ | CL |
|---|---|---|---|---|---|---|
| (h) | (h) | (μg/ml) | (h*ug/mL) | (h*ug/mL) | (mL/kg) | (mL/h/kg) |
| 3.86 | 0.08 | 341.03 | 356.90 | 392.83 | 248.15 | 44.55 |

*Tₘₐₓ and Cₘₐₓ were measured by LC-MS/MS.

Based on analysis of the terminal pharmacokinetics data, the MYDGF-164 fusion protein is cleared in mice with a half-life of 3.86 hour.

In stark contrast, MYDGF without the mucin domain fusion is cleared in vivo with a half-life of merely 15.3 minutes (Korf-Klingebiel 2015). The increased half-life of MYDGF-164 in the fusion is expected to greatly ease the administration of the protein as a therapeutic during disease treatment, and to improve therapeutic outcomes.

In addition, the steady state volume of distribution is 248.15 mL/kg, much higher than the blood volume of mice (70 mL/kg), suggesting that the majority of the MYDGF-164 entered tissues, and the mucin domain fusion did not impact tissue distribution.

Together, the pharmacokinetic behavior strongly suggest that the CD164 mucin domains could be used in protein engineering to enhance the half-life without impacting tissue distributions.

### REFERENCES

Chan et al., Relationship between novel isoforms, functionally important domains, and subcellular distribution of CD164/endolyn. JBC 276(3):2139-52, 2001.
Doyonnas et al., CD164 monoclonal antibodies that block hemopoietic progenitor cell adhesion and proliferation interact with the first mucin domain of the CD164 receptor. The Journal of Immunology 165(2):840-51, 2000.
Fares et al., Design of a long-acting follitropin agonist by fusing the C-terminal sequence of the chorionic gonadotropin beta subunit to the follitropin beta subunit. PNAS USA 89(10):4304-8, 1992.
Fares et al., Development of a long-acting erythropoietin by fusing the carboxyl-terminal peptide of human chorionic gonadotropin β-subunit to the coding sequence of human erythropoietin. Endocrinology 148(10):5081-7, 2007.
Forde et al., Endolyn (CD164) modulates the CXCL12-mediated migration of umbilical cord blood CD133⁺ cells. Blood 109(5):1825-33, 2007.
Hecht et al., Rationale-based engineering of a potent long-acting FGF21 analog for the treatment of type 2 diabetes. PloS One 7(11):e49345, 2012.
Ihrke et al., Endolyn is a mucin-like type I membrane protein targeted to lysosomes by its cytoplasmic tail. Biochemical Journal. 345(2):287-96, 2000.
Korf-Klingebiel et al., Myeloid-derived growth factor (C19orf10) mediates cardiac repair following myocardial infarction. Nature medicine 21(2):140, 2015.
Lee et al., Identification of a role for the sialomucin CD164 in myogenic differentiation by signal sequence trapping in yeast. MCB 21(22):7696-706, 2001.
Tassi et al., Fibroblast Growth Factor Binding Protein 3 (FGFBP3) impacts carbohydrate and lipid metabolism. Scienctific Reports 8:15973, 2018.

All references are incorporated into the places where they are cited.

### Example 5 MYDGF-164 Fusion Protein Promotes HUVECs Proliferation, Migration and Formation Tubes, Resists Apoptosis

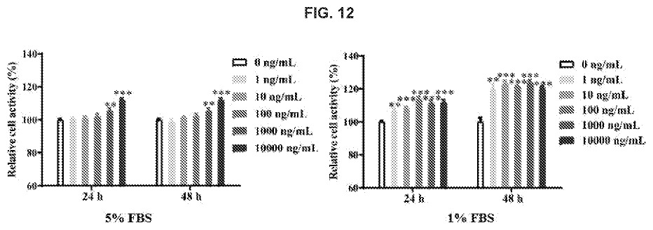

To characterize the proliferative activities on HUVEC cells, MYDGF-164 was added to HUVEC cells that were seeded in 96-well plates and stimulated by the different concentrations of MYDGF-164, in the presence of 5% or 1% FBS (FIG. 12). It was found that MYDGF-164 drove HUVEC cell proliferation in 5% FBS in a dose dependent manner. In 1% FBS, MYDGF-164 had an effect on cell proliferation, but did not increase with the dose increase of MYDGF-164, suggesting that additional growth factors in serum had a synergistic effect with MYDGF-164.

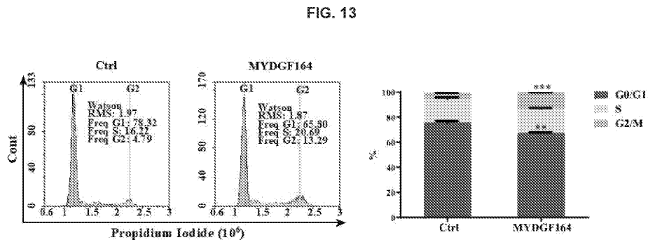

To corroborate that MYDGF-164 can facilitate HUVEC cell replication, flow cytometry was performed to determine the proportion of cells in different phases of cell cycle (FIG. 13). In 1% FBS medium, HUVECs in G0/G1 phase after 24 hours was found to be ˜79%, whereas addition of 1 μg/mL MYDGF-164 decreased G0/G1 phase to ˜65%. Concurrently ˜14% of the total cells entered G2 phase compared to ˜5% of untreated samples. Cells in S phase after treatment was ˜21%, also higher than ˜16% of the untreated samples. These results confirmed that the MYDGF-164 had significant effect upon the cell cycle activities.

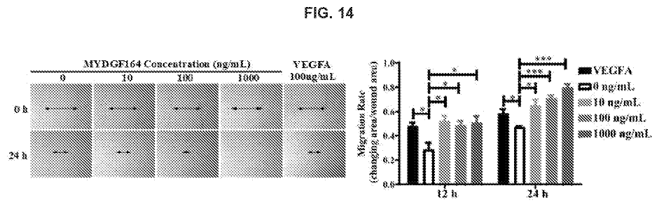

One of the characteristic feature of the endothelial cells is the migratory activities in response to angiogenesis stimuli. The migration behavior of the HUVEC cells in the presence of MYDGF-164 was determined (FIG. 14). In a scratch repair assay, closure of the monolayer of endothelial cells occur through migration. MYDGF-164 promoted migration of HUVEC cells as the scratch closure by migration increased with higher concentration of MYDGF-164 (FIG. 14).

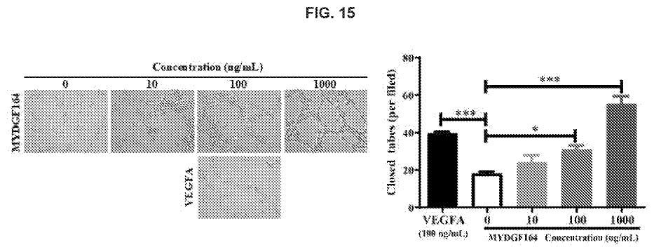

Tube formation by endothelial cells is a critical part of the angiogenesis, and was tested in vitro herein (FIG. 15). Traditionally, the ability of HUVEC cells to form tube-like structures when embedded in growth factor-reduced matrigel (BD, Bioscience) is a measure for angiogenesis potential for endothelial cells. Here, it was demonstrated that MYDGF-164 at various concentration was capable of promoting formation of tube structures as compared to 100 ng/ml VEGFA. To be noted, defined, closed tubes as circular structures seemed to be tighter and more regular-looking for MYDGF-164 compared to VEGFA at the same concentration.

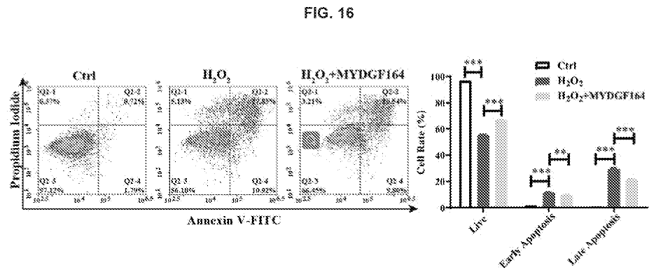

The data further demonstrated that the MYDGF-164 fusion was able to reduce apoptosis triggered by various stimuli. In this assay HUVEC cells were preincubated with 1 μg/mL MYDGF-164 for 24 hours followed by treatment with hydrogen peroxide (400 μmol/L) for another 24 hours (FIG. 16). In the presence of MYDGF-164, propidium and annexin-V double stained cells reduced to 20.54% as opposed to 27.85% of the non-treated sample, suggesting that apoptosis triggered by hydrogen peroxide was significantly reduced (FIG. 16).

### Methods:

Cells line. The HUVECs were purchased from PromoCell (Heidelberg, Germany, primary cells) and cultured in endothelial cell growth medium supplemented with endothelial cell growth supplement (Supplement Mix, PromoCell) and 5% (v/v) FBS and 1% (vol/vol) streptomycin in an atmosphere of 5% CO₂ at 37° C. HUVECs were maintained at a density of 5×10⁶ in T75 flask for three passages at least before experiment (one passage every 3 days).

Cell proliferation analysis. HUVECs were seeded in 96-well plates at 3000 cells/well and cultured for 24 hours, stimulated by the different concentrations of MYDGF-164 with 5% or 1% FBS for 24 hours. Cell proliferation was quantified by CCK-8 assay (Yeasen).

Flow cytometry assay. For cell cycle analysis, the HUVECs were seeded at a density of 2×10⁵ per well in 6-well plates. 1% FBS was presented, and then the vehicle or 1 μg/mL MYDGF-164 were added. 24 hrs after treatment, cells were collected, fixed and stained using Cell Cycle Analysis Kit (Solarbio). The signals were captured by NovoCyte (Agilent BIO) and the data analysis was performed using NovoExpress software. For apoptosis analysis, cells were incubated with 1 μg/mL MYDGF-164 for 24 hours. Then, cells were added with H₂O₂ (400 μmol/L) or cisplatin (DDP, 5 μg/mL) for another 24 hours. Then, the cells were collected and washed with PBS, stained using Cell Apoptosis Assay Kit (Yeasen). Apoptotic and necrotic cells were detected by NovoCyte (Agilent BIO) and the data analysis was performed using NovoExpress software.

Scratch assay. For scratch recovery analysis, the HUVECs were seeded at a density of 1×10⁵ per well in 12-well plates. Supernatant was removed and then a straight line through the cell monolayer was drawn in the middle of each well with a 200 μl tip. The endothelial cell growth medium with different concentrations of MYDGF-164 or 100 ng/ml VEGFA were added. Images were captured at 0, 12 and 24 hrs. The migration rate was analyzed using the Image-Pro-Plus program. The migration rate=changing area/wound area.

Tube formation. Growth factor-reduced Matrigel (BD, Bioscience) was mixed with cell medium at 1:2, and 100 μL/well was plated in 48-well plates, then polymerized at 37° C. for 1 h. 2×10⁴ HUVECs incubated with different concentrations of MYDGF-164 or 100 ng/ml VEGFA in 100 μL were added into each well. After 4-hr incubation, endothelial tubes were photographed. The closed tubes were circular structures completely surrounded by HUVEC cells and the number of closed tubes were counted.

### Example 6 MYDGF-164 Fusion Protein Reduced Myocardial Infarction Area after MI

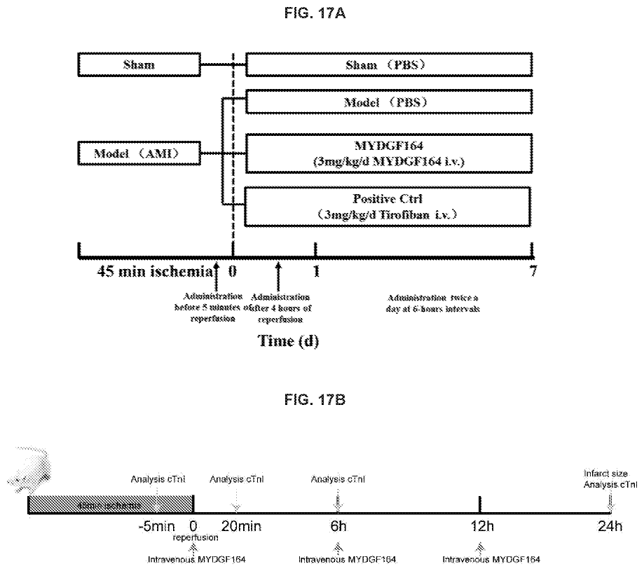

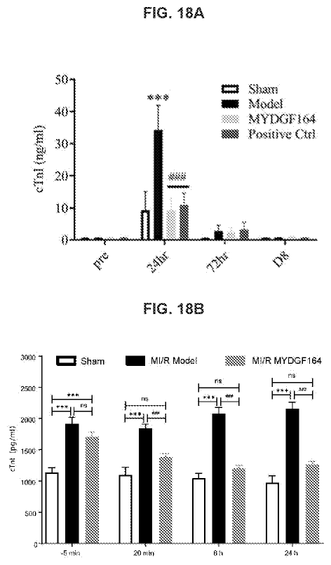

Potential protective function of MYDGF-164 was tested in two rat myocardial ischemia models (FIG. 17A and FIG. 17B). In the first model, 32 six-week-old male SD rats were randomly assigned into sham operation group (sham group) by opening the chest surgically and a model group, which were surgically subjected to 45 min of myocardial ischemia followed by reperfusion. The MYDGF-164 treatment group received MYDGF-164 by tail vein injection at the 5 min before reperfusion. Additional administration MYDGF-164 occurred at 4-hr after reperfusion, then twice a day at 6-hour intervals for the following week. The positive control group were treated with Tirofiban, a non-peptide reversible antagonist of the platelet glycoprotein (GP) IIb/IIIa receptor, and an inhibitor of platelet aggregation. In the second model, the MYDGF-164 treatment is similar except the hearts were assessed 24 hours after ischemic event in order to determine the area at risk.

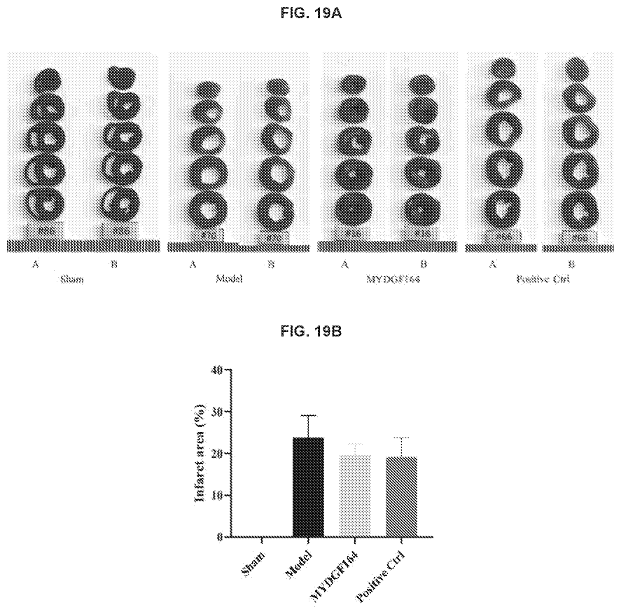

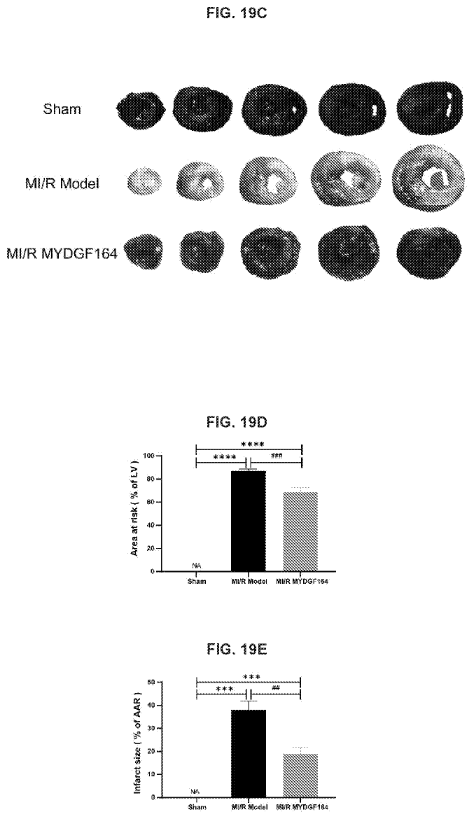

Cardiac-specific troponins I (cTnI) is released by cardiac muscle during myocardial infarction, and measurement of cardiac-specific troponins are extensively used as diagnostic indicator in myocardial infarction and acute coronary syndrome. Blood cTnI was collected before surgical ischemia after surgical operations. Treatment of MYDGF-164 and Tirofiban significantly reduced the serum cTn1 to the levels of the sham-operated rats, demonstrating the cardioprotective effect of MYDGF-164 during experimental myocardial infarction (FIG. 18A an FIG. 18B).

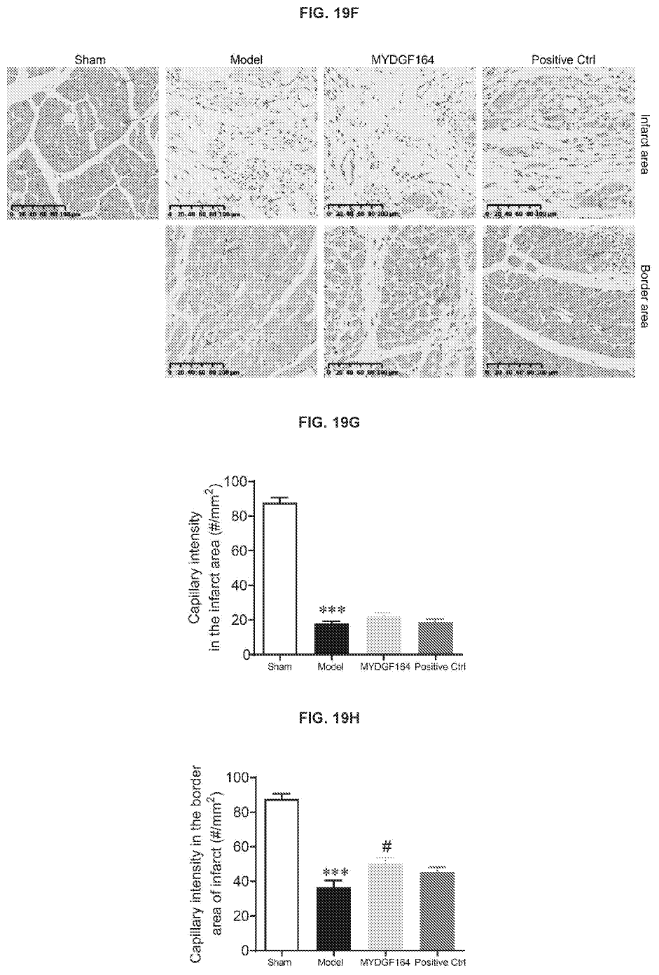

FIG. 19A-19H show MYDGF-164 was capable to decrease the cardiac tissue damages caused by ischemia and to facilitate angiogenesis after reperfusion. Triphenyl tetrazolium chloride (TTC) staining is the method of choice for postmortem determination of myocardial ischemia. Non-infarcted, viable tissue stained red, whereas the infarcted area was left unstained (FIGS. 19A and 19B). Rat hearts were sectioned in 1 mm slices and stained with TTC (FIG. 19A). The results showed that one week after the ischemia the infarct area were reduced by Tirofiban and MYDGF-164 treatment compared to the model group (FIG. 19B).

In the second rat ischemia model, Evans blue staining was added for the postmortem analysis. Ischemic tissues that excluded Evans blue were area at risk for permanent tissue damages. At 24 hours after ischemic event, both area at risk and infarcted area were significantly decreased after the MYDGF-164 treatment (FIG. 19C, FIG. 19D, FIG. 19E).

FIG. 19F-19H show that MYDGF-164 enhanced angiogenesis in the border area between infarcted and non-infarcted tissues. Seven days after the ischemia and treatment, sectioned heart tissues were stained with H&E and capillaries were identified in both infarcted and non-infarcted areas (FIG. 19F, red arrows). The capillary densities in the infarction area were similar for MYDGF164 group and the tirofiban (positive control) group (FIG. 19G). The capillary densities in the border area of infarction was significantly higher after MYDGF164 treatment (FIG. 19H). *** P<0.001 vs. Sham; #P<0.05 vs. Model using one-way ANOVA with Tukey's multiple comparisons test.

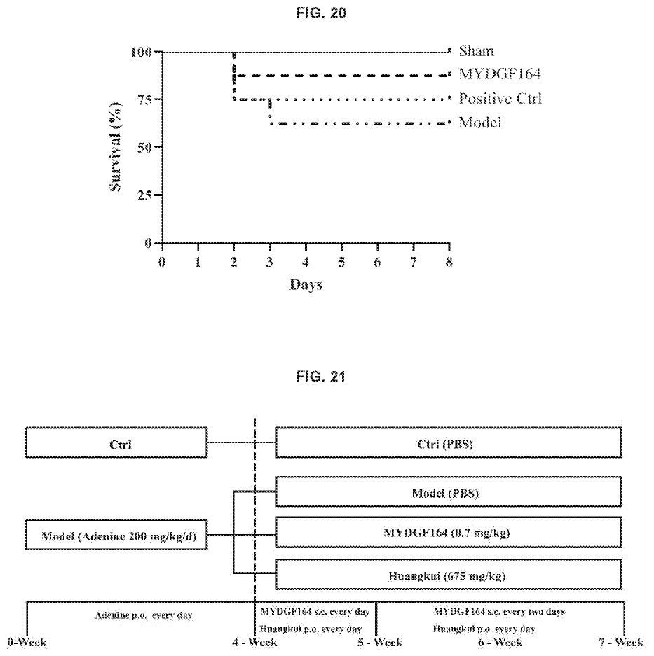

Survival rates of different groups at day 8 were also calculated (FIG. 20). The MYDGF-164 group had the highest survival rate compared to the model and positive control groups. This result is consistent with the overall infarct area data, where the MYDGF-164 group also had the lowest affected myocardial area. The results showed that MYDGF-164 may significantly reduce the death rate of patients with myocardial infarction.

### Example 7 MYDGF-164 Fusion Protein Reduced Adenine-Induced Renal Injury

To investigate whether MYDGF-164 can protect patients from renal injuries, Wistar rats randomly assigned into experimental groups were orally administered 200 mg/kg/d adenine for 4 weeks, followed by treatment by intragastric administration of PBS or 675 mg/kg Huangkui, or by subcutaneous injection of 0.7 mg/kg/day MYDGF-164 for 1 week, followed by 0.7 mg/kg/2 day for additional two weeks. As a negative control, a group of rats were fed with PBS throughout the experiment. During treatment, serum samples were collected weekly to determine creatinine and urea levels according to the manufacturer's protocols (Nanjing Jiancheng Bioengineering Institute). The experiment scheme is depicted in FIG. 21.

After the treatment, the animals were sacrificed and the harvested kidney were weighted. Kidney tissue were fixated in 4% paraformaldehyde (24 hours) and embedded in paraffin. Hematoxylin and eosin (H&E) staining was done according to the standard procedures (Bio-year Tech). The 5-point method was used to determine the degree of kidney lesions. For the immunohistochemistry (IHC) staining, the sections were incubated with the primary antibodies Ki-67 (GB14102; Servicebio, KIM-1(BA3537; BASTER), rat endothelial cell antigen-1 (ab9774; Abcam) overnight, and the corresponding secondary antibody conjugated with HRP then incubated with a DAB solution and nuclear counterstained with haematoxylin. TUNEL signal was stained according to the manufacturer's instructions (Beyotime, Beijing, China).

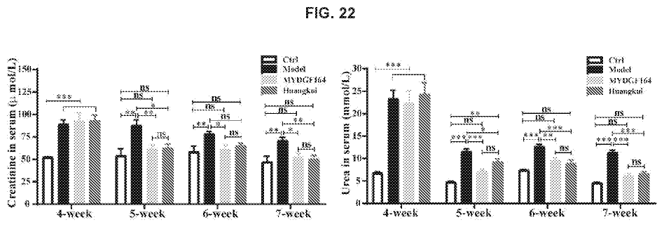

Both urea and creatine are serum markers for kidney function, and their accumulation in serum suggests that kidney secretion are affected, for example by adenine-induced tissue damages (FIG. 22). Serum urea and creatinine were assayed in each group at 4-7 weeks. After 4 weeks of adenine administration, severe renal dysfunction was observed in the model group, as indicated by significantly increased creatinine and urea in serum levels (p<0.001) in comparison with the control group that was fed with PBS only (4-week).

Meanwhile, after one week of subcutaneous administration of the MYDGF-164 protein, the levels of creatinine and urea (5-week) in serum were significantly reduced, with no significant difference to the control group. The effect of MYDGF-164 treatment was comparable to the group where Huangkui was administered intragastrically (5, 6 and 7-week). MYDGF-164 reduced serum urea significantly while Huangkui did not after one week of treatment, suggesting that MYDGF-164 is more potent to treat kidney injuries compared to Huangkui. After the administration of MYDGF-164 and Huangkui in the following two weeks, the creatinine (6-week) and urea (7-week) levels in serum decreased to a level without difference from the control group.

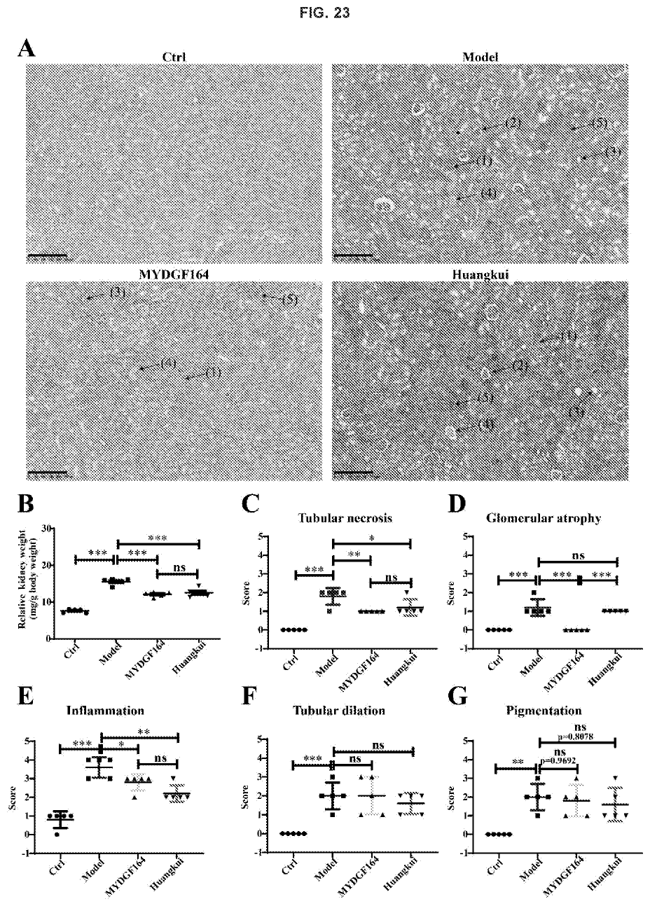

After the treatment regime was completed, all animals were sacrificed. Histopathological examination showed that the kidneys of the model group were pale, and relative kidney weight (mg/g body weight) analysis demonstrated severe hypertrophy (FIG. 23A). The relative kidney weight was significantly reduced compared to the model group after treatment with MYDGF-164 or Huangkui (FIG. 23B), suggesting that the MYDGF-164 protein and Huangkui protected kidney from damages caused by adenine. The 5-point method was used to evaluate the degree of lesion of the kidney tissue. The data showed that, after treatment by MYDGF-164, tubular necrosis (FIG. 23C), glomerular atrophy (FIG. 23D) and inflammation (FIG. 23E) were all reduced when compared to the model group, and the degrees of reduction were similar to that in the Huangkui treatment group.

It is noteworthy that, after the MYDGF-164 treatment, the glomerular atrophy score normalized to the same level as that for the control group (FIG. 23D). However, treatment by MYDGF-164 and Huangkui did not improve tubular dilation (FIG. 23F) and pigmentation (FIG. 23G) after adenine-induced kidney injury.

In the adenine-induced kidney injury model, continued epithelial cell death and subsequent proliferation are undergoing cellular processes underlying the injury and repair. Tubulointerstitial fibrosis as part of the tissue repair processes in chronic kidney disease is associated with a reduction in peritubular capillaries (PTCs).

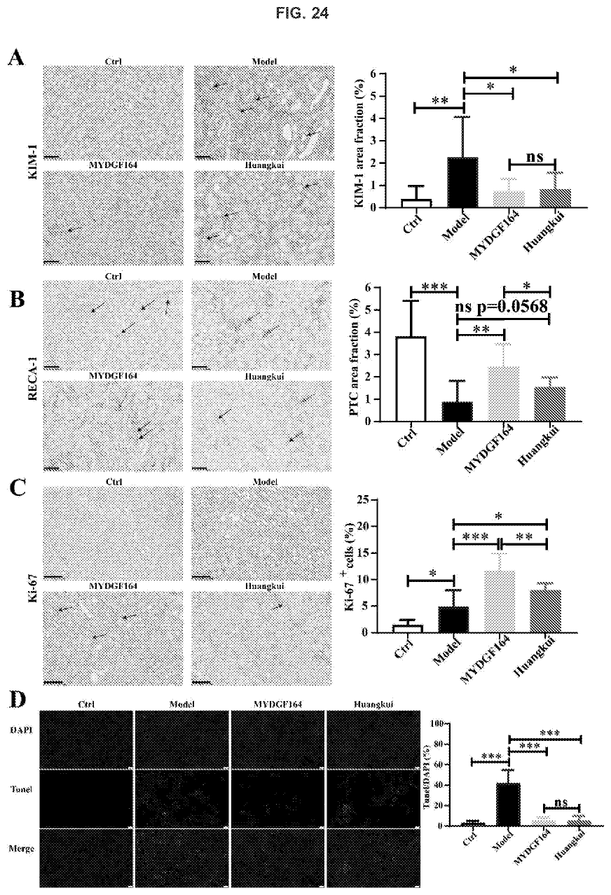

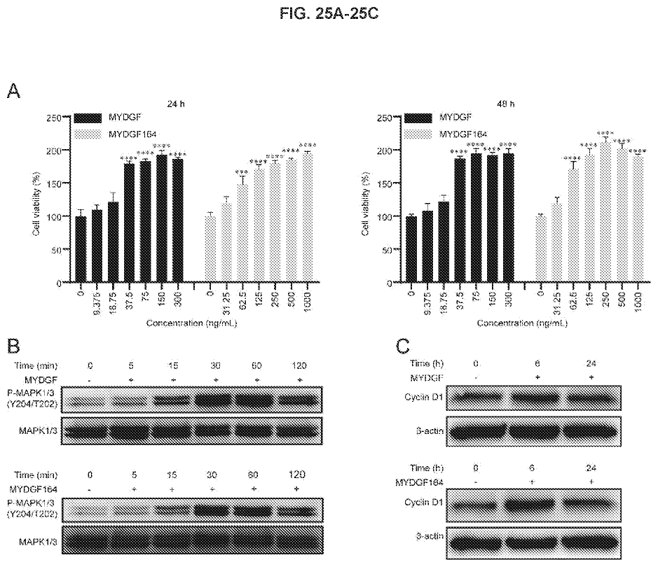

The impact of MYDGF-164 on PTCs apoptosis and proliferation was examined. The level of the biomarker for kidney injury molecule-1 (KIM-1) in the MYDGF-164 and Huangkui treatment groups were significantly decreased (FIG. 24A), suggesting efficient repair of renal tubular injury after the MYDGF-164 and Huangkui treatments.

To assess the MYDGF-164 effect on PTCs after administration, the PTC status was evaluated by staining for RECA-1, a renal endothelial cell marker that illuminates vascular structures in histology analysis. In the model group, many PTCs displayed an abnormal vasculature (i.e., a collapsed lumen structure) (FIG. 24B). In contrast, the filled RECA-1 stained area in the MYDGF-164 group was 2.46% of the total examined tissue area, which were significantly higher than those in the model and Huangkui groups (FIG. 24B). This result indicated that the MYDGF-164 efficiently restored or prevented damages done to the PTCs after withdrawal of the adenine in the kidney injury model.

Cell proliferation is an important process for recovery after renal injury. Kidney sections were stained for Ki-67 to access cellular proliferation. Ki-67 positive cells in the renal tissue increased after MYDGF-164 and Huangkui treatments (FIG. 24C). Moreover, after MYDGF-164 treatment, cell proliferation in the renal tubules and peritubular capillaries increased, suggesting that MYDGF-164 may promote the proliferation of renal tubules epithelial cells, as well the interstitial capillary epithelial cells.

Terminal deoxynucleotidyl transferase-mediated digoxigenin-deoxyuridine nick-end labeling (TUNNEL) is frequently used to characterize apoptotic cells in tissues. The frequency of the observed apoptotic cells in kidney tissue via TUNNEL staining was highly reduced after MYDGF-164 treatment (FIG. 24D), indicating its protective effect against apoptosis/cell death.

### Example 8 HUVECs Proliferation Driven by MYDGF-164 Fusion Protein Correlated with MAPK and Cyclin D1 Activation

Earlier findings suggested that the native MYDGF activated the MAPK1/3 pathway, stimulated phosphorylation of AKT, and increased cyclin D1 expression in HUVECs. To assess whether the fusion to CD164 mucine II domain altered the MYDGF function in this regard, HUVECs (PromoCell, Heidelberg, Germany) were cultured in endothelial cell growth medium supplemented with endothelial cell growth supplement (Supplement Mix, PromoCell) in an atmosphere of 5% CO₂ at 37° C., and were seeded in 96-well plates at 3000 cells/well and cultured for 24 hours, stimulated by the different concentrations of MYDGF164 for 24 or 48 hours. Cell proliferation was quantified by CCK-8 assay (Yeasen). When adjusted for mass differences, there were no differences in proliferative activities between the native MYDGF and MYDGF164 fusion proteins (FIG. 25A). In a Western analysis of HUVECs treated with MYDGF (300 ng/mL) or MYDGF164 (1 μg/mL) fusions, the kinetics of MAPK1/3 phosphorylation were identical for both treatment (FIG. 25B). Furthermore, cyclin D1 expression was also enhanced by MYDGF164 (FIG. 25C). In brief, MYDGF164 fusion most likely maintained the cell signaling functions of MYDGF. Antibodies used for the Western blot analysis were p-MAPK1/3 (Y204/T202, CST, Cat #4370), total-MAPK1/3(CST, Cat #4695), cyclin D1 (CST, Cat #2922), and β-actin (Abways, Cat #AB0035).

### Example 9 MYDGF-164 Fusion Protein In Vitro can Drive HUVECs Migration in Contrast to Nicorandil

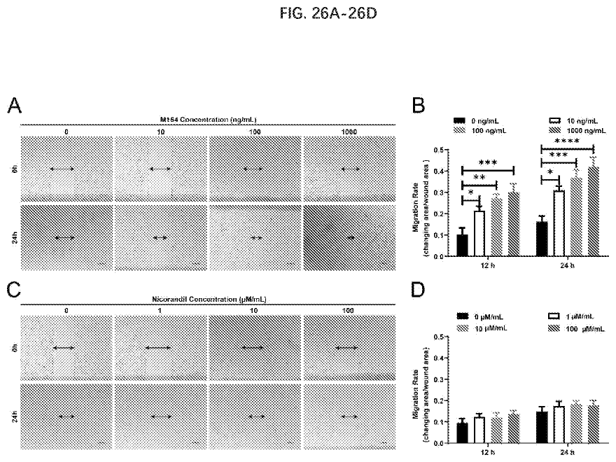

The antianginal agent nicorandil has been applied clinically in treatment of reperfusion-induced damage following coronary angioplasty or thrombolysis. The protective effects of nicorandil had been attributed to the opening of adenosine triphosphate-sensitive potassium (KATP) channel, and potentially the effect of nitric oxide resulting after hydrolysis. It is unclear if nicorandil promoted migratory activities of endothelial cells response to angiogenesis stimuli through nitric oxide. We determined the migration behavior of the HUVECs in presence of MYDGF164 and nicorandil in a scratch repair assay. The closure of the monolayer of endothelial cells occur through migration were enhanced by addition of MYDGF164 in a dose dependent manner (FIG. 26A, B), while addition of nicorandil had no effect upon the migration (FIG. 26C, D). Thus, MYDGF164 may enhance endothelial cell migration, an additional mechanism for tissue repair that is absent when using nicorandil.

### Example 10 MYDGF-164 Fusion Protein is Superior in Promoting Tube Formation Using HUVECs Compared to Nicorandil

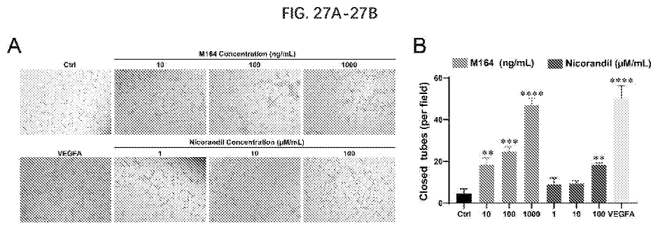

Nitric oxide had been shown to partake in angiogenic processes involving endothelia cells. Tube formation by endothelial cells is a characteristic of angiogenic potentials, and the tube formation promoted by MYDGF164 was compared to that by nitric oxide (FIG. 27A, B). VEGFA was used as a positive control for promotion of tube formation. MYDGF164 demonstrated a dose-dependent activation of tube formation similar to VEGFA. In contrast, endothelial cells treated with highest concentration of nicorandil showed only mild tube formation.

### Example 11 Purified FGF21-164 Fusion Protein

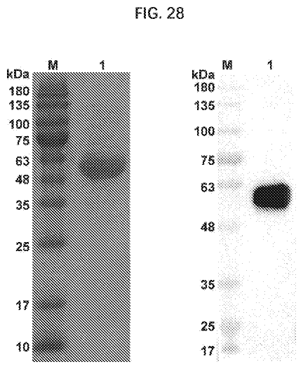

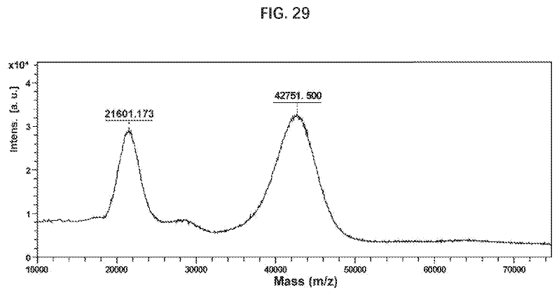

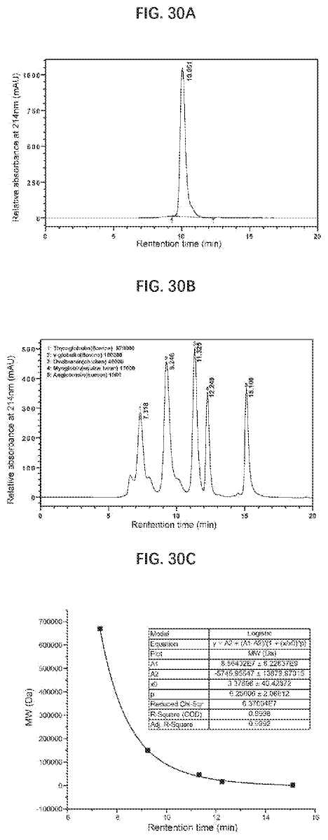

The FGF21-164 fusion proteins were stably expressed in 293F cells, and secreted proteins were purified from the conditioned medium, and the purified protein was analyzed by SDS-PAGE and Western blot analysis (FIG. 28). The predicted molecular masses of mature polypeptide without glycosylation is 24.3 kDa for FGF21-164. Both the SDS-PAGE and Western analysis demonstrated that the FGF21-164 migrated at about 52 kDa, suggesting that the fusion to a CD164 mucin domain accrued additional masses between 20 to 30 kDa.

### Example 12 MALDI-TOF Mass of Purified FGF21-164 Fusion Protein

To determine more accurately the molecular weight mass of the FGF21-164 fusion protein, the purified protein was subjected to MALDI-TOF analysis. While the predicted molecular masses of mature polypeptide without glycosylation is 24.3 kDa for FGF21-164, the MALDI-TOF data (FIG. 29) demonstrated that the mass of singly charged FGF21-164 protein was 42751.5 Dalton, and that of doubly charged was 21601.173 Dalton. Likewise the MYDGF-164 fusion, the added mass is likely due to the glycosylation on the moiety of CD164 mucin domain in the fusion.

### Example 13 SEC Analysis of Purified FGF21-164 Fusion Protein

Analytical SEC remain as a method that determine approximately the hydrodynamic behavior of proteins. The FGF21-164 fusion protein showed an apparent molecular weight equivalent to a globular 88 kDa protein in phosphate buffer (FIG. 30A-C). This observation suggests that the attached mucin domain of the CD164 contributed mostly to the hydrodynamic properties of the FGF21-164 fusion protein. Due to the brush-like conformation of the mucin domain, the FGF21-164 fusion protein unlikely folded into a globular structure. However, it is believed that, similar to Fc fusion or pegylated proteins, the increased hydrodynamic radius of the FGF21-164 should reduce the glomerulus filtration rate, thus increase the half-life of the fusion protein in vivo.

### Example 14 Glycan Analysis of Purified FGF21-164 Fusion Protein

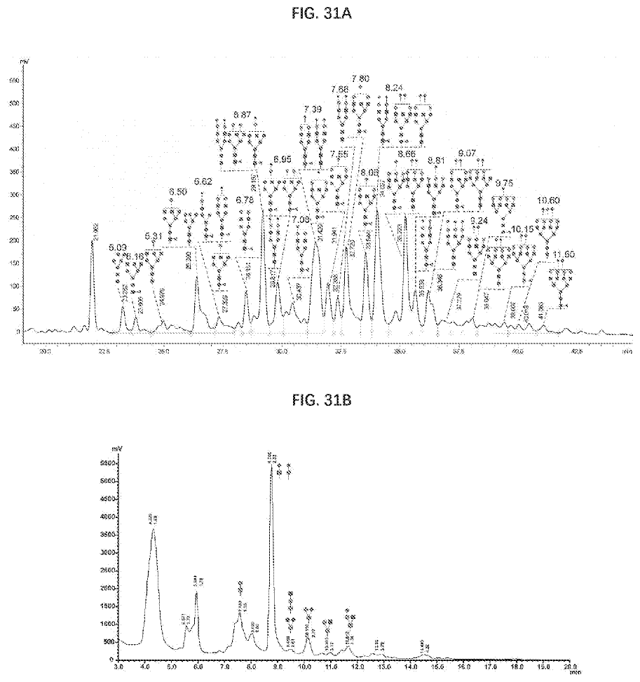

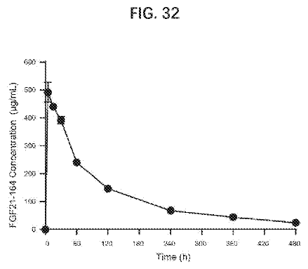

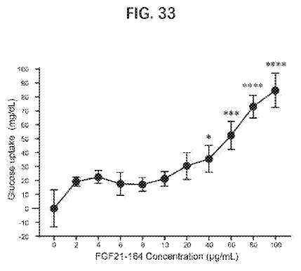

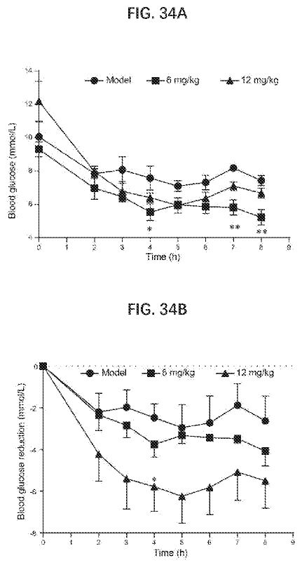

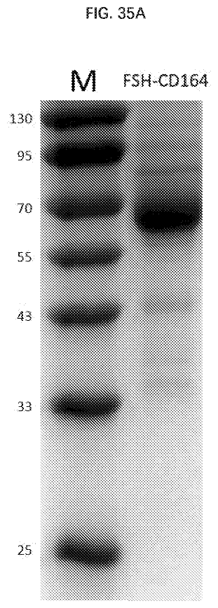

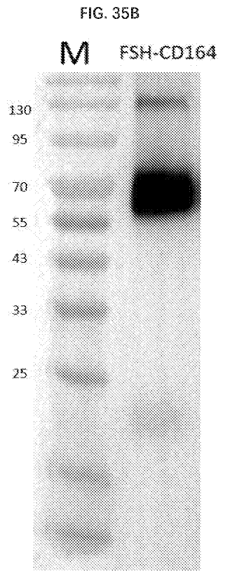

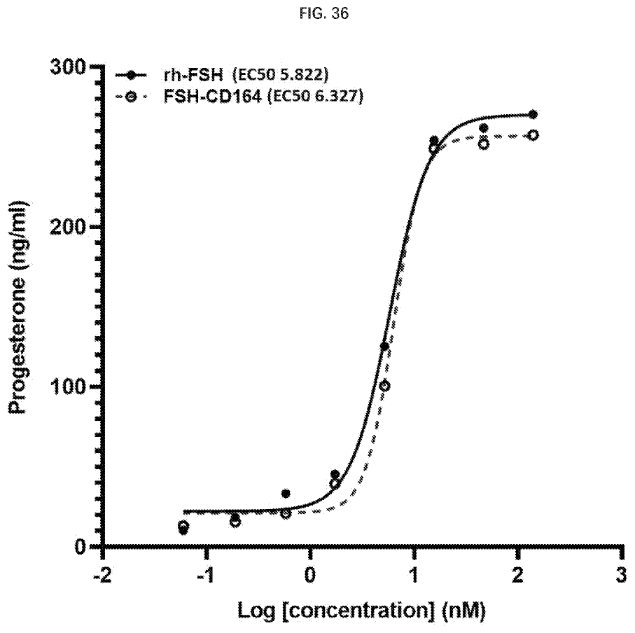

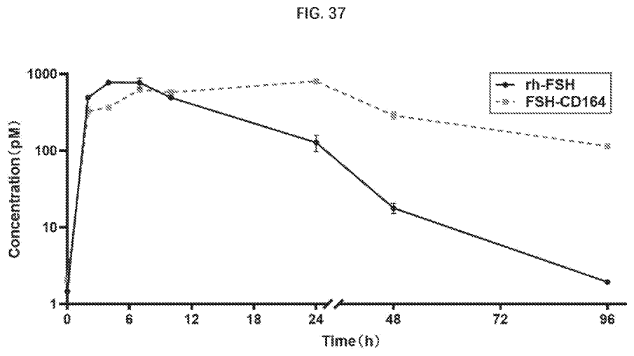

Further analysis was conducted for N-linked glycans by PNGase treatment and 2-AB labeling, followed by analytical UPLC analysis (FIG. 31A). Predominant glycan forms were sialylated biantennary and tri-antennary glycans at 6.87, 7.39, and 8.24 min elution times. Thus the relatively homogeneous biantennary glycan structures found were consistent with the CBB staining and RP-HPLC data (FIG. 3), again suggesting that the N-linked glycosylation of the CD164 mucin domain is highly homogeneous. O-linked glycan were also found (FIG. 31B).

### Example 15 Pharmacokinetic Analysis of Purified FGF21-164 Fusion Protein

The average serum half-life of the FGF21protein was reported previously as 30 minutes. To determine whether the fusion to CD164 mucin domain extended the serum half-life of FGF21, LC-MS method was used to quantify serum concentration of FGF21-164 after intravenous injection into 4 C57/BL6 mice (FIG. 32). The pharmacokinetic parameters of the FGF21-164 was analyzed using non-compartmental model (Table 2). The terminal half-life of FGF21-164 fusion protein was determined to be 2.60±0.335 hour, and the clearance rate (CL) was 21.9±1.22 mL/h/kg, and the stable volume of distribution (Vss) was 64.2±1.78 mL/kg.

TABLE 2
FGF21-164 PK parameters

| | t_{1/2} | AUC₀₋ₜ | AUC_{0-∞} | AUC_%Extrap | CL | Vₛₛ | Cₘₐₓ |
|---|---|---|---|---|---|---|---|
| subject | (h) | (h*μg/mL) | (h*μg/mL) | (%) | (mL/h/kg) | (mL/kg) | (μg/mL) |
| Sample 1 | 2.33 | 1010 | 1090 | 7.86 | 20.8 | 62.7 | 444 |
| Sample 2 | 2.78 | 950 | 1050 | 9.69 | 21.6 | 66.0 | 482 |
| Sample 3 | 2.97 | 947 | 1060 | 10.3 | 21.5 | 65.4 | 549 |
| Sample 4 | 2.30 | 892 | 961 | 7.14 | 23.6 | 62.6 | 541 |
| N | 4 | 4 | 4 | 4 | 4 | 4 | 4 |
| Mean | 2.60 | 949 | 1040 | 8.74 | 21.9 | 64.2 | 504 |
| SD | 0.335 | 46.4 | 55.6 | 1.47 | 1.22 | 1.78 | 49.9 |
| Geometric | 2.58 | 948 | 1040 | 8.64 | 21.9 | 64.1 | 502 |
| Mean | | | | | | | |

The concentration of murine internal YLY peptide was approximately 50 pg/mL˜2 ng/mL, and far less than the lower limit of quantitation of 4.00 μg/mL.

### Example 15 Glucose Uptake Stimulated by Purified FGF21-164 Fusion Protein

It is known that the FGF21 binds to FGFR1 and βKlotho and functions as an agonist to stimulate glucose uptake by adipocytes. FGF21 FGF21-164 fusion protein demonstrated a dose-dependent stimulation of the glucose uptake by 3T3-L1 adipocytes (FIG. 33)

### Example 15 Purified FSH-164 Fusion Protein

The FSH-164 fusion proteins were stably expressed in CHO cells, and secreted proteins were purified from the conditioned medium, and the purified protein was analyzed by SDS-PAGE and Western blot analysis (FIG. 35). The predicted molecular masses of mature, heterodimeric FSH polypeptide is approximately 45 kDa including glycosylation. Both the SDS-PAGE and Western analysis demonstrated that the FSH-164 migrated at about 70 kDa, suggesting that the fusion to a CD164 mucin domain accrued additional masses between 20 to 30 kDa.

### Example 16 Purified FSH-164 Fusion Protein were Active in Stimulating Progesterone Biosynthesis

FSH stimulation promotes progesterone synthesis and output from human granulosa cells without luteinization. KGN cell line is a granulosa-like tumor cell line that can be used to test in vitro FSH function. The purified FSH-164 fusion proteins were tested on the KGN cells (FIG. 36). The FSH-164 stimulated progesterone biosynthesis in the KGN cells with EC50 equal to 6.3 nM, similar to that of FSH (EC50=5.8) in the same assay.

### Example 17 Purified FSH-164 Fusion Protein Demonstrate Extended PK In Vivo

FSH164 fusion protein clearly showed extension of half-life in vivo when compared to recombinant FSH (FIG. 37). The half-life of purified FSH164 was approximately 26.7 hours (Table 3) compared to that of 10 hours for the recombinant FSH. Theoretically the 2.5 fold increase of the half-life for FSH is sufficient to support one-dose treatment regimen per each ovulation cycle.

TABLE 3
Summary of PK data of FSH164

| | | | | | Vz/F | CL/F |
|---|---|---|---|---|---|---|
| t_{1/2} | Tₘₐₓ | Cₘₐₓ | AUC₀₋ₜ | AUC_{0-∞} | (μmol)/ | (μmol)/ |
| (h) | (h) | (pmole/L) | (pmole/L*h) | (pmol/L*h) | (pmol/L) | (pmol/L)/h |
| 26.68 | 24 | 804 | 36798.5 | 41202.11 | 0.047 | 0.00122 |

We claim:

1. A fusion protein, comprising: (1) a polypeptide comprising an amino acid sequence having at least 90% identity to SEQ ID NO: 1 or 2, and (2) a heterologous polypeptide.

2. The fusion protein of claim 1, wherein the polypeptide consists of the amino acid sequence of SEQ ID NO: 1 or 2.

3. The fusion protein of claim 1 or 2, wherein (1) is C-terminal to (2), optionally, (1) is SEQ ID NO: 2.

4. The fusion protein of claim 1 or 2, wherein (1) is N-terminal to (2), optionally, (1) is SEQ ID NO: 1.

5. The fusion protein of any one of claims 1-4, further comprising (3) a second polypeptide comprising an amino acid sequence having at least 90% identity to SEQ ID NO: 1 or 2, wherein (1) and (3) each comprising a different one of SEQ ID NO: 1 and 2.

6. The fusion protein of any one of claims 1-5, comprising the polypeptide of SEQ ID NO: 1 fused N-terminal to the heterologous polypeptide, and the polypeptide of SEQ ID NO: 2 fused C-terminal to the heterologous polypeptide.

7. The fusion protein of any one of claims 1-6, wherein said heterologous polypeptide is a therapeutic polypeptide.

8. The fusion protein of claim 7, wherein the therapeutic polypeptide has a (human or mouse) serum half-life that is at least 10%, 20%, 30%, 40%, 50%, 60%, 70%, 80%, 85%, 90%, or 95% less than that of the fusion protein.

9. The fusion protein of any one of claims 1-8, wherein the heterologous polypeptide is fibroblast growth factor 21 (FGF21), follicle-stimulating hormone (FSH), myeloid-derived growth factor (MYDGF), fibroblast growth factor binding protein 3 (FGFBP3), natriuretic peptides B, cholecystokinin, glucagon-like peptide-1 (GLP-1), gonadotropin-releasing hormone, secretin, leuprorelin, enfuvirtide, glucagon, bivalirudin, sermorelin, corticotropin tetracosapeptide, insulin-like growth factor (IGF), parathyroid hormone, or amylin.

10. The fusion protein of any one of claims 1-9, which comprises an O- and/or an N-linked glycosylation.

11. The fusion protein of any one of claims 1-10, which comprises sialylation.

12. The fusion protein of any one of claims 1-11, further comprising a linker peptide between (1) and (2).

13. The fusion protein of any one of claims 1-12, wherein (1) is C-terminal to (2) and is optionally SEQ ID NO: 2, and the heterologous polypeptide is MYDGF or a functional fragment thereof.

14. The fusion protein of any one of claims 1-13, wherein the fusion protein has an amino acid sequence having at least 90% identity to SEQ ID NO: 3.

15. The fusion protein of claim 14, which has the amino acid sequence of SEQ ID NO: 3.

16. A polynucleotide encoding the fusion protein of any one of claims 1-15.

17. The polynucleotide of claim 16, which is codon-optimized for expression in a target host cell.

18. The polynucleotide of claim 17, wherein the target host cell is a human cell, a rodent cell (e.g., a mouse cell), or a non-human mammalian cell.

19. A vector comprising the polynucleotide of any one of claims 16-18.

20. The vector of claim 19, which is an expression vector.

21. The vector of claim 19 or 20, which is a plasmid.

22. A host cell comprising the fusion protein of any one of claims 1-15, the polynucleotide of any one of claims 16-18, or the vector of any one of claims 19-21.

23. The host cell of claim 22, which is a tissue culture cell.

24. The host cell of claim 22 or 23, which is a CHO cell (e.g., CHO-K1 or derivative thereof), or a HEK293 cell or derivative thereof.

25. A pharmaceutical composition comprising a therapeutically effective amount of the fusion protein of any one of claims 1-15, the polynucleotide of any one of claims 16-18, or the vector of any one of claims 19-21, and a pharmaceutically acceptable carrier or excipient.

26. The pharmaceutical composition of claim 25, which is formulated for intravenous injection.

27. A method of enhancing serum half-life for a protein, comprising fusing the protein to a polypeptide comprising an amino acid sequence having at least 90% identity to SEQ ID NO: 1 or 2.

28. The method of claim 27, wherein the polypeptide consists of the amino acid sequence of SEQ ID NO: 1 or 2.

29. The method of claim 27 or 28, wherein the protein is fused N-terminal to SEQ ID NO: 2.

30. The method of any one of claims 27-29, wherein the protein is fused to the polypeptide via a linker polypeptide.

31. A method of treating a disease, disorder, or condition in a subject in need thereof, the method comprises administering to the subject a therapeutically effective amount of the fusion protein of any one of claims 1-15, the polynucleotide of any one of claims 16-18, or the vector of any one of claims 19-21, wherein the disease, disorder, or condition is treatable by said heterologous polypeptide.

32. The method of claim 31, wherein the disease, disorder, or condition is selected from the group consisting of a tissue injury, a cardiovascular disease, an inflammatory disease or disorder, and a kidney disease.

33. The method of claim 32, wherein the tissue injury is an acute injury such as myocardio infarction or stroke.

34. The method of claim 32, wherein the tissue injury is a chronic injury such as diabetic injury to kidney.

35. The method of claim 32, wherein the cardiovascular disease is selected from the group consisting of myocardial infarction, arteriosclerosis, hypertension, angina pectoris, hyperlipidemia, and heart failure.

36. The method of claim 32, wherein the inflammatory disease or disorder is selected form the group consisting of Type I diabetes, Type II diabetes, pancreatitis, nonalcoholic fatty liver disease (NAFLD), and nonalcoholic steatohepatitis (NASH).

37. The method of claim 31, wherein the disease or disorder is a kidney disease.

38. The method of any one of claims 31-37, wherein the subject is a human.
